SEQUENCE LISTING (3 sequences)

SEQ ID NO: 1 - DNA, 19 bp, Artificial Sequence
  Synthetic
gaccttcagaaggttgctg

SEQ ID NO: 2 - DNA, 19 bp, Artificial Sequence
  Synthetic
gccagattctgccgaacca

SEQ ID NO: 3 - DNA, 19 bp, Artificial Sequence
  Synthetic
gtgcagtatcctctgacag

(19) United States
(12) Patent Application Publication
(10) Pub. No.: US 2011/0092452 A1
(43) Pub. Date: Apr. 21, 2011

(54) COMPOSITIONS AND METHODS FOR DIAGNOSING AND TREATING PANCREATIC CANCER

(75) Inventors: Diane M. Simeone, Ann Arbor, MI (US); Chenwei LI, Windsor (CA)

(73) Assignee: THE REGENTS OF THE UNIVERSITY OF MICHIGAN, Ann Arbor, MI (US)

(21) Appl. No.: 12/920,755

(22) PCT Filed: Mar. 5, 2009
(86) PCT No.: PCT/US2009/036199

§ 371 Date: Dec. 21, 2010

Related U.S. Application Data
(60) Provisional application No. 61/033,889, filed on Mar. 5, 2008.

(51) Int. Cl. A61K 31/70; G01N 33/574; C12N 5/00; A61K 31/505; A61K 31/4545; A61K 31/403; A61P 35/00

(52) U.S. Cl. 514/49; 435/7.23; 435/325; 514/274; 514/318; 514/414

(57) ABSTRACT

The present invention provides compositions and methods for treating and diagnosing the presence of solid tumor stem cells in a patient (e.g., based on the presence of c-Met). In certain embodiments, the presence or absence of solid tumor stem cells (e.g., pancreatic solid tumor stem cells) can be determined through the use of a single cells surface marker (e.g., c-Met). The present invention also provides methods for screening candidate compounds (e.g., c-Met antagonists) for the ability to inhibit the tumorigenisis of pancreatic solid tumor stem cells.

The present application claims priority to U.S. Provisional Application Ser. No. 61/033,889, filed Mar. 5, 2008, which is herein incorporated by reference in its entirety.

### FIELD OF THE INVENTION

The present invention relates to compositions and methods for treating and diagnosing the presence of solid tumor stem cells in a patient (e.g., based on the presence of c-Met). In certain embodiments, the presence or absence of solid tumor stem cells (e.g., pancreatic solid tumor stem cells) can be determined through the use of a single cells surface marker (e.g., c-Met). The present invention also provides methods for screening candidate compounds (e.g., c-Met antagonists) for the ability to inhibit the tumorigenisis of solid tumor stem cells.

### BACKGROUND

Pancreatic adenocarcinoma is a highly lethal disease which is usually diagnosed in an advanced state for which there are little/no effective therapies. It has the worst prognosis of any major malignancy (3% 5 year survival) and is the fourth most common cause of cancer death per year in the United States, with an annual incidence rate approximating the annual death rate of 31,000 people. Despite advances in surgical and medical therapy, little impact has been made on the mortality rate of this disease. One of the major hallmarks of pancreatic cancer is its extensive local tumor invasion and early systemic dissemination. The molecular basis for these characteristics of pancreatic cancer is incompletely understood.

Attempts to better understand the molecular characteristics of pancreatic cancer have focused on studying gene and protein expression profiles of samples of pancreatic cancer. However, these types of studies have not taken into account the heterogeneity of cancer cells within a particular tumor. A practical consequence of this tumor cell heterogeneity is that strategies for inducing cell death must address the unique survival mechanisms of each different cell type within the malignant population.

What is needed are improved compositions and methods for understanding, detecting, and treating cancer having heterogeneous cell populations.

### SUMMARY OF THE INVENTION

The present invention provide compositions and methods for treating and diagnosing the presence of solid tumor stem cells in a patient (e.g., based on the presence of c-Met). In certain embodiments, the presence or absence of solid tumor stem cells (e.g., pancreatic solid tumor stem cells) can be determined through the use of a single cells surface marker (e.g., c-Met). The present invention also provides methods for screening candidate compounds (e.g., c-Met antagonists) for the ability to inhibit the tumorigenisis of pancreatic solid tumor stem cells.

In some embodiments, the present invention provides methods of detecting solid tumor stem cells comprising; a) providing: i) a tissue sample taken from a tumor of a subject, and ii) an antigen binding protein (e.g., an antibody, or antibody fragment), specific for the c-Met protein; and b) contacting the tissue sample with the antigen binding protein (e.g., antibody, or antibody fragment), under conditions such that the presence or absence of c-Met+ solid tumor stem cells are detected.

In particular embodiments, the contacting comprises employing standard immunohistochemistry techniques and reagents that are well known in the art. In certain embodiments, the antigen binding protein (e.g., the antibody, or antibody fragment), is conjugated to a signal molecule. In further embodiments, the signal molecule comprises a fluorescent molecule. In further embodiments, the signal molecule comprises an enzyme that can catalyze a color producing reaction in the presence of a colorimetric substrate. In other embodiments, the method further comprises contacting the sample with a secondary antibody, or secondary antibody fragment, specific for the antibody or antibody fragment. In certain embodiments, the secondary antibody, or secondary antibody fragment, comprises a signal molecule. In further embodiments, no other proteins or nucleic acids are assayed in order to determine the presence or absence of the c-Met+ solid tumor stem cells. In particular embodiments, the tumor is a pancreatic tumor.

In some embodiments, the present invention provides methods of enriching for a population of solid tumor stem cells comprising: a) disassociating a solid tumor to generate disassociated cells; b) contacting the disassociated cells with a reagent that binds c-Met; and c) selecting cells that bind to the reagent under conditions such that a population enriched for solid tumor stem cells is generated. In particular embodiments, the population enriched for solid tumor stem cells is generated without employing reagents for any other solid tumor stem cell markers besides the reagent that binds c-Met. In certain embodiments, no additional reagents are employed in order to generate the population enriched for solid tumor stem cells (i.e., a single reagent specific for c-Met is employed). In particular embodiments, the solid tumor is a pancreatic solid tumor. In further embodiments, the reagent is an antibody or antibody fragment (e.g., the eBioclone 97 monoclonal antibody and those described in Pat. Pub. 20050054019, which is herein incorporated by reference in its entirety). In some embodiments, the reagent is conjugated to a fluorochrome or magnetic particles. In other embodiments, selecting cells is performed by flow cytometry, fluorescence activated cell sorting, panning, affinity column separation, or magnetic selection. In particular embodiments, the present invention provides an enriched population of solid tumor stem cells isolated by the method described above.

In some embodiments, the present invention provides an isolated population of cancer stem cells that are: a) tumorigenic; and b) c-Met+. In certain embodiments, the cancer stem cells are pancreatic cancer stem cells. In other embodiments, the population comprises at least 60% cancer stem cells and less than 40% non-tumorigenic tumor cells (e.g., at least 60%, at least 65%, at least 70%, at least 75%, at least 80%, or at least 85% cancer stem cells). In certain embodiments, the cellular composition comprising cancer stem cells and non-tumorigenic tumor cells is obtained without employing reagents for any other solid tumor stem cell markers besides reagents that bind c-Met. In further embodiments, the cancer stem cells are enriched at least two-fold compared to unfractionated non-tumorigenic tumor cells (e.g., enriched at least 2-fold, 3-fold, 4-fold, 5-fold, . . . 10-fold, . . . 100-fold, . . . 1000-fold enriched).

In particular embodiments, the present invention provides methods of diagnosing the presence of pancreatic solid tumor stem cells in a patient comprising: a) contacting a sample from the patient comprising tumor cells with a reagent that binds c-Met, and b) identifying the presence or absence of pancreatic solid tumor stem cells in the sample, wherein presence of cells that are c-Met+ indicates that pancreatic solid tumor stem cells are present. In certain embodiments, the presence or absence of pancreatic solid tumor stem cells in sample are identified without employing reagents for any other solid tumor stem cell markers besides the reagent that binds c-Met.

In additional embodiments, the present invention provides methods for obtaining from a tumor a cellular composition comprising cancer stem cells and non-tumorigenic tumor cells, wherein at least 60% are tumorigenic stem cells and 40% or less are non-tumorigenic tumor cells, the method comprising: a) obtaining a dissociated mixture of tumor cells from a tumor; b) separating the mixture of tumor cells into a first fraction comprising at least 60% cancer stem cells and 40% or less non-tumorigenic tumor cells and a second fraction of tumor cells depleted of cancer stem cells wherein the separating is by contacting the mixture with a reagent against c-Met; and c) demonstrating the first fraction to be tumorigenic by serial injection into a first host animal and the second fraction to be non-tumorigenic by serial injection into a second host animal. In certain embodiments, the separating is performed by flow cytometry, fluorescence activated cell sorting (FACS), panning, affinity chromatography or magnetic selection. In particular embodiments, the separating is performed by fluorescence activated cell sorters (FACS) analysis.

In some embodiments, the present invention provides methods of treating a patient with pancreatic cancer comprising: administering a c-Met inhibitor to the patient such that the number of pancreatic stem cells expressing c-Met is reduced or eliminated in the subject. In other embodiments, the methods further comprise administering a chemotherapeutic selected from the group consisting of: 5-fluorouracil (5-FU); Gemcitabine (Gemzar); and Capecitabine. In certain embodiments, a second agent (e.g., standard pancreatic cancer chemotherapeutic) is administered with, or at about the same time, as the c-Met inhibitor. In particular embodiments, the c-Met inhibitor comprises siRNA or antisense specific for c-Met. The nucleic acid sequence of human c-Met is known (accession number NM 000245, herein incorporated by reference). As such, one of skill in the art can design antisense or siRNA sequences (e.g., by entering the c-Met sequence into a computer program designed to provide such sequences). In other embodiments, the c-Met inhibitor is PHA-665752 or PF-02341066. In particular embodiments, the method further comprise, prior to the administering, a step of contacting a sample from the patient comprising pancreatic tumor cells with a reagent that binds c-Met. In further embodiments, the methods further comprise identifying the patient as having c-Met+ pancreatic solid tumor stem cells.

In some embodiments, the present invention provides methods for selecting a treatment for a patient having a solid tumor, comprising: (a) obtaining a sample from the patient; (b) identifying the presence of a c-Met+ solid tumor stem cell in the sample; and (c) selecting a treatment for the patient that targets c-Met+ solid tumor stem cells. In further embodiments, the c-Met+ solid tumor stem cells are pancreatic solid tumor stem cells.

In other embodiments, the present invention provides methods of reducing or eliminating tumorigenic cells in a subject, comprising: administering a c-Met antagonist, or c-Met signaling pathway antagonist, to the subject under conditions such that at least a portion of the tumorigenic cells are killed, inhibited from proliferating, or from causing metastasis. In certain embodiments, the tumorigenic cells are pancreatic cells.

In additional embodiments, the present invention provides methods for screening a compound, comprising: a) exposing a sample comprising a c-Met+ pancreatic cell to a candidate anti-neoplastic compound, wherein the candidate anti-neoplastic compound comprises a c-Met antagonist or a c-Met signaling pathway antagonist; and b) detecting a change in the cell in response to the compound. In some embodiments, the sample comprises a non-adherent mammosphere. In further embodiments, the c-Met antagonist, or c-Met signaling pathway, antagonist comprises an antibody or antibody fragment. In certain embodiments, the c-Met antagonist is a tyrosine kinase inhibitor. In some embodiments, the detecting comprises detecting cell death of the tumorigenic breast cell. In further embodiments, the methods further comprise identifying the candidate anti-neoplastic agent as capable of killing tumorigenic cells.

In particular embodiments, the present invention provides methods for determining the capability of a test compound to inhibit tumorigenesis of pancreatic solid tumor stem cells comprising: a) obtaining enriched pancreatic solid tumor stem cells, wherein the pancreatic solid tumor stem cells: i) are enriched at least two-fold compared to unfractionated tumor cells; and ii) express c-Met; b) exposing a first set, but not a second set, of the pancreatic solid tumor stem cells to a test compound; c) injecting the first set of the solid tumor stem cells into a first host animal and injecting the second set of solid tumor stem cells into a second host animal; and d) comparing a tumor, if present, in the first animal with a tumor formed in the second animal in order to determine if the test compound inhibits tumor formation. In other embodiments, the test compound is a c-Met inhibitor, or a c-Met pathway inhibitor.

In certain embodiments, the present invention provides methods for determining the capability of a test compound to inhibit tumorigenesis of pancreatic solid tumor stem cells comprising: a) obtaining enriched pancreatic solid tumor stem cells, wherein the solid tumor stem cells: i) are enriched at least two-fold compared to unfractionated tumor cells; and ii) express c-Met; b) injecting the pancreatic solid tumor stem cells into first and second host animals; c) treating the first host animal with a test compound, and not treating the second host animal with the test compound; and d) comparing a tumor, if present, in the first host animal with a tumor formed in the second host animal in order to determine if the test compound inhibits tumor formation. In particular embodiments, the test compound is a c-Met inhibitor or a c-Met pathway inhibitor.

In further embodiments, the present invention provides methods for determining the capability of a test compound to inhibit tumorigenesis of pancreatic solid tumor stem cells comprising: a) obtaining a sample comprising at least 60% pancreatic solid tumor stem cells, wherein the solid tumor stem cells express c-Met; b) injecting the pancreatic solid tumor stem cells into first and second host animals; c) treating the first host animal with a test compound, and not treating the second host animal with the test compound; and d) comparing a tumor, if present, in the first animal with a tumor formed in the second animal in order to determine if the test compound inhibits tumor formation. In other embodiments, the test compound is a c-Met inhibitor or a c-Met pathway inhibitor.

### DESCRIPTION OF FIGURES

FIG. 1A. Flow cytometry was used to isolate subpopulations of human pancreatic cancer cells which were tested for tumorigenicity in NOD/SCID mice. Cells were stained with antibodies against c-Met, H2K and DAPI. Dead cells and mouse cells were eliminated from the analyses. The plot depicted is a representative example of c-Met staining of human pancreatic cancer cells from an individual patient, with the frequency of the boxed tumorigenic cancer cell population as a percentage of cancer cells in the specimen shown.

FIG. 1B shows tumor formation in NOD/SCID mice injected with c-Met+ cancer cells.

FIG. 2 shows results from Example 1 where tumor sphere formation was observed in c-Met+ (FIG. 2A) but not c-Met− (FIG. 2B) sorted single cells.

### DEFINITIONS

To facilitate an understanding of the present invention, a number of terms and phrases are defined below:

As used herein, the terms “anticancer agent,” “conventional anticancer agent,” or “cancer therapeutic drug” refer to any therapeutic agents (e.g., chemotherapeutic compounds and/or molecular therapeutic compounds), radiation therapies, or surgical interventions, used in the treatment of cancer (e.g., in mammals).

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered. It is intended that the terms “drug” and “chemotherapeutic agent” encompass anti-hyperproliferative and antineoplastic compounds as well as other biologically therapeutic compounds.

An “effective amount” is an amount sufficient to effect beneficial or desired results. An effective amount can be administered in one or more administrations.

As used herein, the term “administration” refers to the act of giving a drug, prodrug, antibody, or other agent, or therapeutic treatment to a physiological system (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

“Coadministration” refers to administration of more than one chemical agent or therapeutic treatment (e.g., radiation therapy) to a physiological system (e.g., a subject or in vivo, in vitro, or ex vivo cells, tissues, and organs). “Coadministration” of the respective chemical agents (e.g. c-Met signaling pathway antagonist) and therapeutic treatments (e.g., radiation therapy) may be concurrent, or in any temporal order or physical combination.

As used herein, the term “regression” refers to the return of a diseased subject, cell, tissue, or organ to a non-pathological, or less pathological state as compared to basal nonpathogenic exemplary subject, cell, tissue, or organ. For example, regression of a tumor includes a reduction of tumor mass as well as complete disappearance of a tumor or tumors.

As used herein the term, “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reactions that occur within a natural environment.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “subject” or “patient” refers to organisms to be treated by the methods of the present invention. Such organisms include, but are not limited to, humans and veterinary animals (dogs, cats, horses, pigs, cattle, sheep, goats, and the like). In the context of the invention, the term “subject” or “patient” generally refers to an individual who will receive or who has received treatment.

The term “diagnosed,” as used herein, refers to the recognition of a disease by its signs and symptoms or genetic analysis, pathological analysis, histological analysis, and the like.

As used herein, the term “antisense” is used in reference to nucleic acid sequences (e.g., RNA, phosphorothioate DNA) that are complementary to a specific RNA sequence (e.g., mRNA). Included within this definition are natural or synthetic antisense RNA molecules, including molecules that regulate gene expression, such as small interfering RNAs or micro RNAs.

The term “test compound” or “candidate compound” refers to any chemical entity, pharmaceutical, drug, and the like, that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment or prevention. In preferred embodiments, “test compounds” are anticancer agents. In particularly preferred embodiments, “test compounds” are anticancer agents that induce apoptosis in cells.

As used herein, the term “antigen binding protein” refers to proteins which bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibodies, various host animals can be immunized by injection with the peptide corresponding to the desired epitope including, but not limited to, rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 (1975)), as well as the trioma technique, the human B-cell hybridoma technique (See e.g., Kozbor et al., Immunol. Today, 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 (1989)) to allow rapid and easy identification of monoclonal Fab fragments with the desired specificity.

Antibody fragments that contain the idiotype (antigen binding region) of the antibody molecule can be generated by known techniques. For example, such fragments include, but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen-binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

As used herein, the term “modulate” refers to the activity of a compound to affect (e.g., to promote or retard) an aspect of the cellular function including, but not limited to, cell growth, proliferation, invasion, angiogenesis, apoptosis, and the like.

### DETAILED DESCRIPTION OF THE INVENTION

The present invention provide compositions and methods for treating and diagnosing the presence of solid tumor stem cells in a patient (e.g., based on the presence of c-Met). In certain embodiments, the presence or absence of solid tumor stem cells (e.g., pancreatic solid tumor stem cells) can be determined through the use of a single cells surface marker (e.g., c-Met). The present invention also provides methods for screening candidate compounds (e.g., c-Met antagonists) for the ability to inhibit the tumorigenisis of pancreatic solid tumor stem cells.

The present invention relates to compositions and methods for characterizing, regulating, diagnosing, and treating cancer. For example, the present invention provides compositions and methods for inhibiting tumorigenesis of certain classes of cancer cells (e.g., pancreatic cells), and preventing metastasis (e.g., using c-Met signaling pathway antagonists). The present invention also provides systems and methods for identifying compounds that regulate tumorigenesis. For example, the present invention provides methods for identifying tumorigenic cells and diagnosing diseases (e.g., hyperproliferative diseases) or biological events (e.g., tumor metastasis) associated with the presence of tumorigenic cells. In particular, the present invention identifies classes of cells within cancers that are tumorigenic and provides detectable characteristics of such cells (e.g., expression of c-Met), such that their presence can be determined, for example, in choosing whether to submit a subject to a medical intervention, selecting an appropriate treatment course of action, monitoring the success or progress of a therapeutic course of action (e.g., in a drug trial or in selecting individualized, ongoing therapy), or screening for new therapeutic compounds or therapeutic targets.

In some embodiments, the expression of c-Met or a c-Met signaling pathway component is used to identify tumorigenic cells. Regulators of c-Met signaling pathway components also find use in research, drug screening, and therapeutic methods. For example, c-Met signaling pathway antagonists and antagonists of the c-Met signaling pathways find use in preventing or reducing cell proliferation, hyperproliferative disease development or progression, and cancer metastasis. In some embodiments, antagonists are utilized following removal of a solid tumor mass to help reduce proliferation and metastasis of remaining hyperproliferative cells.

The present invention is not limited to any particular type of tumorigenic cell type, nor is the present invention limited by the nature of the compounds or factors used to regulate tumorigenesis. Thus, while the present invention is illustrated below using pancreatic, skilled artisans will appreciate that the present invention is not limited to these illustrative examples. For example, it is contemplated that are variety of neoplastic conditions benefit from the teachings of the present invention, including, but not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

I. c-Met and the c-Met Signaling Pathway

The c-Met protein (also known as hepatocyte growth factor receptor; or HGFR) is a 145 kDa receptor tyrosine kinase (RTK) normally expressed by epithelial cells of the brain, kidney, liver and other tissues. Binding of its ligand, Hepatocyte Growth Factor (HGF), triggers receptor autophosphorylation, and activation of several downstream effectors including the mitogen-activated protein kinases ERK-1 and ERK-2, and PLC-γ. Activation of the c-Met signal transduction pathway leads to multiple cellular responses including cell motility, scattering, proliferation, survival and angiogenesis. Mutations in human c-Met have been implicated in the development of several malignancies.

### II. Tumorigenic Cancer Cells

Solid tumors are composed of heterogeneous populations of cancer cells that differ in their ability to form new tumors. Cancer cells that have the ability to form tumors (i.e., tumorigenic cancer cells) and cancer cells that lack this capacity (i.e., non-tumorigenic cancer cells) can be distinguished based on phenotype (Al-Hajj, et al., Proc Natl Acad Sci USA 100, 3983-8 (2003); Pat. Pub. 20020119565; Pat. Pub. 20040037815; Pat. Pub. 20050232927; WO05/005601; Pat. Pub. 20050089518; U.S. application Ser. No. 10/864,207; Al-Hajj et al., Oncogene, 2004, 23:7274; and Clarke et al., Ann Ny Acad. Sci., 1044:90, 2005, all of which are herein incorporated by reference in their entireties for all purposes).

The art has previously identified a highly tumorigenic population of primary pancreatic cancer cells expressing the cell surface markers CD44, CD24, and ESA (Li et al., Cancer Res., 2007, Fe. 1; 67(3):1030-7, herein incorporated by reference), which represents 0.2-0.8% of pancreatic cancer cells. These cells exhibit the stem cell properties of self-renewal, the ability to produce differentiated progeny, and increased expression of developmental signaling pathways. The present invention identifies c-Met (HGF receptor) as a cell surface marker of pancreatic cancer stem cells that is important in both their identification and function.

The cellular responses to c-Met stimulation by hepatocyte growth factor (HGF) are important in mediating a wide range of biological activities in cancer cells including cell motility, invasion, migration and eventual metastasis (Maulik et al., Ctyokine Growth Factor Rev. Feb. 13, 2002(1):41-59, herein incorporated by reference). c-Met is over-expressed in a variety of cancers including lung, breast, colorectal, prostate, pancreatic, head and neck, gastric, hepatocellular, ovarian, renal, glioma, melanoma and sarcoma. The activation of c-Met is transient in normal physiological conditions, while it is constitutively activated in many human cancer cells. The mechanisms by which c-Met becomes activated in human cancers includes overexpression, activating mutations of other pathway components, or HGF-dependent autocrine/paracrine activation.

A link between c-Met and pancreatic cancer stem cells has not been previously made, although c-Met has been described as a putative marker of normal pancreatic stem cells (Suzuki et al., Diabetes, 2004, 53:2143-2152). As shown in the Example below, a subpopulation of highly tumorigenic cancer cells expressing the single cell surface marker c-Met has been identified. This was shown using both in vivo xenograft and in vitro tumor sphere assays demonstrating equivalent or higher tumorigenic potential when compared to CD44+CD24+ESA+ cells from human pancreatic cancers.

### III. Detection of Solid Tumor Stem Cell Cancer Markers

In some embodiments, the present invention provides methods for detection of expression of stem cell cancer markers (e.g., pancreatic cancer stem cell cancer markers). In some embodiments, expression is measured directly (e.g., at the RNA or protein level). In some embodiments, expression is detected in tissue samples (e.g., biopsy tissue). In other embodiments, expression is detected in bodily fluids (e.g., including but not limited to, plasma, serum, whole blood, mucus, and urine).

It is noted that the present invention allows the use of a single marker (c-Met) to detect solid tumor stem cells (e.g., from the pancreas), whereas the art indicates that multiple markers are to be employed. For example, for detecting pancreatic solid tumor stem cells, the art indicates that three markers are to be used (CD44, CD24, and ESA). Using a triple marker scheme may be difficult to translate to the clinic to measure cancer stem cells number and/or the effectiveness of therapeutic targeting of pancreatic cancer stem cells. In general, to measure CD44+, CD24+, and ESA+ cells, one would have to use cell sorting (e.g., using FACS). This may be difficult or impossible to do with a small biopsy sample or fine need aspirate. The use of c-Met as a single marker allows the use of immunohistochemistry on small biopsy samples or fine need aspirates (or the like) to measure pancreatic cancer stem cells.

The present invention further provides panels and kits for the detection of markers. In some embodiments, the presence of a stem cell cancer marker is used to provide a prognosis to a subject. The information provided is also used to direct the course of treatment. For example, if a subject is found to have a marker indicative of a solid tumor stem cell (e.g., c-Met), additional therapies (e.g., radiation therapies) can be started at an earlier point when they are more likely to be effective (e.g., before metastasis). In addition, if a subject is found to have a tumor that is not responsive to certain therapy, the expense and inconvenience of such therapies can be avoided.

In some embodiments, the present invention provides a panel for the analysis of a plurality of markers (e.g., the combination of c-Met and at least one of CD44, CD24, and ESA). The panel allows for the simultaneous analysis of multiple markers correlating with carcinogenesis and/or metastasis. Depending on the subject, panels can be analyzed alone or in combination in order to provide the best possible diagnosis and prognosis. Markers for inclusion on a panel are selected by screening for their predictive value using any suitable method, including but not limited to, those described in the illustrative examples below.

1. Detection of RNA

In some embodiments, detection of solid tumor stem cell cancer markers are detected by measuring the expression of corresponding mRNA in a tissue sample (e.g., pancreatic cancer tissue). mRNA expression can be measured by any suitable method, including but not limited to, those disclosed below.

In some embodiments, RNA is detected by Northern blot analysis. Northern blot analysis involves the separation of RNA and hybridization of a complementary labeled probe.

In still further embodiments, RNA (or corresponding cDNA) is detected by hybridization to an oligonucleotide probe). A variety of hybridization assays using a variety of technologies for hybridization and detection are available. For example, in some embodiments, TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR(RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

2. Detection of Protein

In other embodiments, gene expression of stem cell cancer markers is detected by measuring the expression of the corresponding protein or polypeptide (e.g., c-Met expression). Protein expression can be detected by any suitable method. In some embodiments, proteins are detected by immunohistochemistry. In other embodiments, proteins are detected by their binding to an antibody raised against the protein. The accession number for human c-Met protein is NP_{—}000236, the sequence of which is herein incorporated by reference. The generation of antibodies is described below.

Antibody binding is detected by techniques known in the art (e.g., radioimmunoassay, ELISA (enzyme linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference.

3. cDNA Microarray Technology

cDNA microarrays are composed of multiple (usually thousands) of different cDNAs spotted (usually using a robotic spotting device) onto known locations on a solid support, such as a glass microscope slide. The cDNAs are typically obtained by PCR amplification of plasmid library inserts using primers complementary to the vector backbone portion of the plasmid or to the gene itself for genes where sequence is known. PCR products suitable for production of microarrays are typically between 0.5 and 2.5 kB in length. Full length cDNAs, expressed sequence tags (ESTs), or randomly chosen cDNAs from any library of interest can be chosen. ESTs are partially sequenced cDNAs as described, for example, in Hillier, et al., 1996, 6:807-828. Although some ESTs correspond to known genes, frequently very little or no information regarding any particular EST is available except for a small amount of 3′ and/or 5′ sequence and, possibly, the tissue of origin of the mRNA from which the EST was derived. As will be appreciated by one of ordinary skill in the art, in general the cDNAs contain sufficient sequence information to uniquely identify a gene within the human genome. Furthermore, in general the cDNAs are of sufficient length to hybridize, selectively, specifically or uniquely, to cDNA obtained from mRNA derived from a single gene under the hybridization conditions of the experiment.

In a typical microarray experiment, a microarray is hybridized with differentially labeled RNA, DNA, or cDNA populations derived from two different samples. Most commonly RNA (either total RNA or poly A+ RNA) is isolated from cells or tissues of interest and is reverse transcribed to yield cDNA. Labeling is usually performed during reverse transcription by incorporating a labeled nucleotide in the reaction mixture. Although various labels can be used, most commonly the nucleotide is conjugated with the fluorescent dyes Cy3 or Cy5. For example, Cy5-dUTP and Cy3-dUTP can be used. cDNA derived from one sample (representing, for example, a particular cell type, tissue type or growth condition) is labeled with one fluorophore while cDNA derived from a second sample (representing, for example, a different cell type, tissue type, or growth condition) is labeled with the second fluorophore. Similar amounts of labeled material from the two samples are cohybridized to the microarray. In the case of a microarray experiment in which the samples are labeled with Cy5 (which fluoresces red) and Cy3 (which fluoresces green), the primary data (obtained by scanning the microarray using a detector capable of quantitatively detecting fluorescence intensity) are ratios of fluorescence intensity (red/green, R/G). These ratios represent the relative concentrations of cDNA molecules that hybridized to the cDNAs represented on the microarray and thus reflect the relative expression levels of the mRNA corresponding to each cDNA/gene represented on the microarray.

Each microarray experiment can provide tens of thousands of data points, each representing the relative expression of a particular gene in the two samples. Appropriate organization and analysis of the data is of key importance, and various computer programs that incorporate standard statistical tools have been developed to facilitate data analysis. One basis for organizing gene expression data is to group genes with similar expression patterns together into clusters. A method for performing hierarchical cluster analysis and display of data derived from microarray experiments is described in Eisen et al., 1998, PNAS 95:14863-14868. As described therein, clustering can be combined with a graphical representation of the primary data in which each data point is represented with a color that quantitatively and qualitatively represents that data point. By converting the data from a large table of numbers into a visual format, this process facilitates an intuitive analysis of the data. Additional information and details regarding the mathematical tools and/or the clustering approach itself can be found, for example, in Sokal & Sneath, Principles of numerical taxonomy, xvi, 359, W.H. Freeman, San Francisco, 1963; Hartigan, Clustering algorithms, xiii, 351, Wiley, New York, 1975; Paull et al., 1989, J. Natl. Cancer Inst. 81:1088-92; Weinstein et al. 1992, Science 258:447-51; van Osdol et al., 1994, J. Natl. Cancer Inst. 86:1853-9; and Weinstein et al., 1997, Science, 275:343-9.

Further details of the experimental methods used in the present invention are found in the Example below. Additional information describing methods for fabricating and using microarrays is found in U.S. Pat. No. 5,807,522, which is herein incorporated by reference. Instructions for constructing microarray hardware (e.g., arrayers and scanners) using commercially available parts. Additional discussions of microarray technology and protocols for preparing samples and performing microrarray experiments are found in, for example, DNA arrays for analysis of gene expression, Methods Enzymol, 303:179-205, 1999; Fluorescence-based expression monitoring using microarrays, Methods Enzymol, 306: 3-18, 1999; and M. Schena (ed.), DNA Microarrays: A Practical Approach, Oxford University Press, Oxford, UK, 1999.

4. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject can visit a medical center to have the sample obtained and sent to the profiling center, or subjects can collect the sample themselves and directly send it to a profiling center. Where the sample comprises previously determined biological information, the information can be directly sent to the profiling service by the subject (e.g., an information card containing the information can be scanned by a computer and the data transmitted to a computer of the profiling center using an electronic communication system). Once received by the profiling service, the sample is processed and a profile is produced (e.g., expression data), specific for the diagnostic or prognostic information desired for the subject.

The profile data is then prepared in a format suitable for interpretation by a treating clinician. For example, rather than providing raw expression data (e.g. examining a number of the markers), the prepared format can represent a diagnosis or risk assessment for the subject, along with recommendations for particular treatment options. The data can be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject can chose further intervention or counseling based on the results. In some embodiments, the data is used for research use. For example, the data can be used to further optimize the inclusion or elimination of markers as useful indicators of a particular condition or stage of disease.

5. Kits

In yet other embodiments, the present invention provides kits for the detection and characterization of cancer (e.g. for detecting one or more of the markers, or for modulating the activity of a peptide expressed by one or more of markers). In some embodiments, the kits contain antibodies specific for a cancer marker, in addition to detection reagents and buffers. In other embodiments, the kits contain reagents specific for the detection of mRNA or cDNA (e.g., oligonucleotide probes or primers). In some embodiments, the kits contain all of the components necessary and/or sufficient to perform a detection assay, including all controls, directions for performing assays, and any necessary software for analysis and presentation of results.

Another embodiment of the present invention comprises a kit to test for the presence of the polynucleotides or proteins. The kit can comprise, for example, an antibody for detection of a polypeptide or a probe for detection of a polynucleotide. In addition, the kit can comprise a reference or control sample; instructions for processing samples, performing the test and interpreting the results; and buffers and other reagents necessary for performing the test. In other embodiments the kit comprises pairs of primers for detecting expression of one or more of the genes of the solid tumor stem cell gene signature. In other embodiments the kit comprises a cDNA or oligonucleotide array for detecting expression of one or more of the genes of the solid tumor stem cell gene signature.

6. In Vivo Imaging

In some embodiments, in vivo imaging techniques are used to visualize the expression of cancer markers in an animal (e.g., a human or non-human mammal). For example, in some embodiments, cancer marker mRNA (e.g., c-Met mRNA) or protein (e.g., c-Met protein) is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of the present invention are useful in the diagnosis of cancers that express the solid tumor stem cell cancer markers of the present invention (e.g., in pancreatic cancer). In vivo imaging is used to visualize the presence of a marker indicative of the cancer. Such techniques allow for diagnosis without the use of an unpleasant biopsy. The in vivo imaging methods of the present invention are also useful for providing prognoses to cancer patients. For example, the presence of a marker indicative of cancer stem cells can be detected. The in vivo imaging methods of the present invention can further be used to detect metastatic cancers in other parts of the body.

In some embodiments, reagents (e.g., antibodies) specific for c-Met are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin One 9:631-640 [1991]) have described the use of this agent in detecting tumors in patients suspected of having pancreatic cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents can also be used, but the 1-(p-carboxymethoxybenzyl) EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement can be achieved by effecting radiolabeling in the presence of the specific stem cell cancer marker of the present invention, to insure that the antigen binding site on the antibody will be protected.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

IV. c-Met Antibodies and Antibody Fragments

The present invention provides isolated antibodies and antibody fragments against c-Met. The antibody, or antibody fragment, can be any monoclonal or polyclonal antibody that specifically recognizes c-Met. Exemplary c-Met antibodies are found in U.S. Pat. Pub. 20050054019, which is herein incorporated by reference. In some embodiments, the present invention provides monoclonal antibodies, or fragments thereof (e.g., Fab or F(ab′)2 fragments), that specifically bind to c-Met. In some embodiments, the monoclonal antibodies, or fragments thereof, are chimeric or humanized antibodies that specifically bind to c-Met. In other embodiments, the monoclonal antibodies, or fragments thereof (e.g., Fab or F(ab′)2 fragments), are human antibodies that specifically bind to c-Met.

The antibodies against c-Met find use in the experimental, diagnostic and therapeutic methods described herein. In certain embodiments, the antibodies of the present invention are used to detect the expression of a pancreatic cancer stem cell marker protein in biological samples such as, for example, a patient tissue biopsy, pleural effusion, or blood sample. Tissue biopsies can be sectioned and protein detected using, for example, immunofluorescence or immunohistochemistry. Alternatively, individual cells from a sample are isolated, and protein expression detected on fixed or live cells by FACS analysis. Furthermore, the antibodies can be used on protein arrays to detect expression of a pancreatic cancer stem cell marker, for example, on tumor cells, in cell lysates, or in other protein samples. In other embodiments, the antibodies of the present invention are used to inhibit the growth of tumor cells by contacting the antibodies with tumor cells either in vitro cell based assays or in vivo animal models. In still other embodiments, the antibodies are used to treat cancer in a human patient by administering a therapeutically effective amount of an antibody against a pancreatic cancer stem cell marker.

Polyclonal antibodies can be prepared by any known method. Polyclonal antibodies can be raised by immunizing an animal (e.g. a rabbit, rat, mouse, donkey, etc) by multiple subcutaneous or intraperitoneal injections of the relevant antigen (a purified peptide fragment, full-length recombinant protein, fusion protein, etc) optionally conjugated to keyhole limpet hemocyanin (KLH), serum albumin, etc. diluted in sterile saline and combined with an adjuvant (e.g. Complete or Incomplete Freund's Adjuvant) to form a stable emulsion. The polyclonal antibody is then recovered from blood, ascites and the like, of an animal so immunized. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc.

Monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay such as radioimmunoassay (RIA) or enzyme-linked immunosorbent assay (ELISA) can then be propagated either in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

Alternatively monoclonal antibodies can also be made using recombinant DNA methods as described in U.S. Pat. No. 4,816,567. The polynucleotides encoding a monoclonal antibody are isolated, such as from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries as described (McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified in a number of different manners using recombinant DNA technology to generate alternative antibodies. In one embodiment, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity, affinity, etc. of a monoclonal antibody.

In some embodiments, of the present invention the monoclonal antibody against a pancreatic cancer stem cell marker is a humanized antibody. Humanized antibodies are antibodies that contain minimal sequences from non-human (e.g., murine) antibodies within the variable regions. Such antibodies are used therapeutically to reduce antigenicity and HAMA (human anti-mouse antibody) responses when administered to a human subject. In practice, humanized antibodies are typically human antibodies with minimum to no non-human sequences. A human antibody is an antibody produced by a human or an antibody having an amino acid sequence corresponding to an antibody produced by a human.

Humanized antibodies can be produced using various techniques known in the art. An antibody can be humanized by substituting the CDR of a human antibody with that of a non-human antibody (e.g. mouse, rat, rabbit, hamster, etc.) having the desired specificity, affinity, and capability (Jones et al., 1986, Nature, 321:522-525; Riechmann et al., 1988, Nature, 332:323-327; Verhoeyen et al., 1988, Science, 239:1534-1536). The humanized antibody can be further modified by the substitution of additional residue either in the Fv framework region and/or within the replaced non-human residues to refine and optimize antibody specificity, affinity, and/or capability.

Human antibodies can be directly prepared using various techniques known in the art. Immortalized human B lymphocytes immunized in vitro or isolated from an immunized individual that produce an antibody directed against a target antigen can be generated (See, for example, Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373). Also, the human antibody can be selected from a phage library, where that phage library expresses human antibodies (Vaughan et al., 1996, Nature Biotechnology, 14:309-314; Sheets et al., 1998, PNAS, 95:6157-6162; Hoogenboom and Winter, 1991, J. Mol. Biol., 227:381; Marks et al., 1991, J. Mol. Biol., 222:581). Humanized antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

This invention also encompasses bispecific antibodies that specifically recognize a pancreatic cancer stem cell marker. Bispecific antibodies are antibodies that are capable of specifically recognizing and binding at least two different epitopes.

Bispecific antibodies can be intact antibodies or antibody fragments. Techniques for making bispecific antibodies are common in the art (Millstein et al., 1983, Nature 305:537-539; Brennan et al., 1985, Science 229:81; Suresh et al, 1986, Methods in Enzymol. 121:120; Traunecker et al., 1991, EMBO J. 10:3655-3659; Shalaby et al., 1992, J. Exp. Med. 175:217-225; Kostelny et al., 1992, J. Immunol. 148:1547-1553; Gruber et al., 1994, J. Immunol. 152:5368; and U.S. Pat. No. 5,731,168).

In certain embodiments of the invention, it may be desirable to use an antibody fragment, rather than an intact antibody, to increase tumor penetration, for example. Various techniques are known for the production of antibody fragments. Traditionally, these fragments are derived via proteolytic digestion of intact antibodies (for example Morimoto et al., 1993, Journal of Biochemical and Biophysical Methods 24:107-117 and Brennan et al., 1985, Science, 229:81). However, these fragments are now typically produced directly by recombinant host cells as described above. Thus Fab, Fv, and scFv antibody fragments can all be expressed in and secreted from E. coli or other host cells, thus allowing the production of large amounts of these fragments. Alternatively, such antibody fragments can be isolated from the antibody phage libraries discussed above. The antibody fragment can also be linear antibodies as described in U.S. Pat. No. 5,641,870, for example, and can be monospecific or bispecific. Other techniques for the production of antibody fragments will be apparent to the skilled practitioner.

It may further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

The present invention further embraces variants and equivalents which are substantially homologous to the chimeric, humanized and human antibodies, or antibody fragments thereof, set forth herein. These can contain, for example, conservative substitution mutations, i.e. the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

The invention also pertains to immunoconjugates comprising an antibody conjugated to a cytotoxic agent. Cytotoxic agents include chemotherapeutic agents, growth inhibitory agents, toxins (e.g., an enzymatically active toxin of bacterial, fungal, plant, or animal origin, or fragments thereof), radioactive isotopes (i.e., a radioconjugate), etc. Chemotherapeutic agents useful in the generation of such immunoconjugates include, for example, methotrexate, adriamicin, doxorubicin, melphalan, mitomycin C, chlorambucil, daunorubicin or other intercalating agents. Enzymatically active toxins and fragments thereof that can be used include diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies including 212Bi, ,131I, 131In, 90Y, and 186Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). Conjugates of an antibody and one or more small molecule toxins, such as a calicheamicin, maytansinoids, a trichothene, and CC1065, and the derivatives of these toxins that have toxin activity, can also be used.

In some embodiments the antibody of the invention contains human Fc regions that are modified to enhance effector function, for example, antigen-dependent cell-mediated cyotoxicity (ADCC) and/or complement dependent cytotoxicity (CDC). This can be achieved by introducing one or more amino acid substitutions in an Fc region of the antibody. For example, cysteine residue(s) can be introduced in the Fc region to allow interchain disulfide bond formation in this region to improve complement-mediated cell killing and antibody-dependent cellular cytotoxicity (ADCC) (Caron et al., 1992, J. Exp Med. 176:1191-1195; Shopes, 1992, Immunol. 148:2918-2922). Homodimeric antibodies with enhanced anti-tumor activity can also be prepared using heterobifunctional cross-linkers as described in Wolff et al., 1993, Cancer Research 53:2560-2565. Alternatively, an antibody can be engineered which has dual Fc regions (Stevenson et al., 1989, Anti-Cancer Drug Design 3:219-230).

### V. Drug Screening

In some embodiments, the present invention provides drug screening assays (e.g., to screen for anticancer drugs). The screening methods of the present invention utilize stem cell cancer markers (e.g., c-Met and c-Met signaling pathway proteins) identified using the methods of the present invention. For example, in some embodiments, the present invention provides methods of screening for compounds that alter (e.g., increase or decrease) the expression of c-Met. In some embodiments, candidate compounds are antisense agents or siRNA agents (e.g., oligonucleotides) directed against cancer markers. In other embodiments, candidate compounds are antibodies that specifically bind to a stem cell cancer marker of the present invention. In certain embodiments, libraries of compounds of small molecules are screened using the methods described herein.

In one screening method, candidate compounds are evaluated for their ability to alter stem cell cancer marker expression by contacting a compound with a cell expressing a stem cell cancer marker and then assaying for the effect of the candidate compounds on expression. In some embodiments, the effect of candidate compounds on expression of a cancer marker gene is assayed by detecting the level of cancer marker mRNA expressed by the cell. mRNA expression can be detected by any suitable method. In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein. In some embodiments, other changes in cell biology (e.g., apoptosis) are detected.

Specifically, the present invention provides screening methods for identifying modulators, i.e., candidate or test compounds or agents (e.g., proteins, peptides, peptidomimetics, peptoids, small molecules or other drugs) which bind to, or alter the signaling or function associated with the cancer markers of the present invention, have an inhibitory (or stimulatory) effect on, for example, stem cell cancer marker expression or cancer markers activity, or have a stimulatory or inhibitory effect on, for example, the expression or activity of a cancer marker substrate. Compounds thus identified can be used to modulate the activity of target gene products (e.g., stem cell cancer marker genes, such as c-Met) either directly or indirectly in a therapeutic protocol, to elaborate the biological function of the target gene product, or to identify compounds that disrupt normal target gene interactions. Compounds which inhibit the activity or expression of cancer markers are useful in the treatment of proliferative disorders, e.g., cancer, particularly metastatic cancer or eliminating or controlling tumor stem cells to prevent or reduce the risk of cancer.

In one embodiment, the invention provides assays for screening candidate or test compounds that are substrates of a cancer markers protein or polypeptide or a biologically active portion thereof. In another embodiment, the invention provides assays for screening candidate or test compounds that bind to or modulate the activity of a cancer marker protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds can be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

In one embodiment, an assay is a cell-based assay in which a cell that expresses a stem cell cancer marker protein or biologically active portion thereof is contacted with a test compound, and the ability of the test compound to the modulate cancer marker's activity is determined. Determining the ability of the test compound to modulate stem cell cancer marker activity can be accomplished by monitoring, for example, changes in enzymatic activity. The cell, for example, can be of mammalian origin.

The ability of the test compound to modulate cancer marker binding to a compound, e.g., a stem cell cancer marker substrate, can also be evaluated. This can be accomplished, for example, by coupling the compound, e.g., the substrate, with a radioisotope or enzymatic label such that binding of the compound, e.g., the substrate, to a cancer marker can be determined by detecting the labeled compound, e.g., substrate, in a complex.

Alternatively, the stem cell cancer marker is coupled with a radioisotope or enzymatic label to monitor the ability of a test compound to modulate cancer marker binding to a cancer markers substrate in a complex. For example, compounds (e.g., substrates) can be labeled with 121I, 35S, 14C or 3H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a stem cell cancer marker substrate) to interact with a stem cell cancer marker with or without the labeling of any of the interactants can be evaluated. For example, a microphysiorneter can be used to detect the interaction of a compound with a cancer marker without the labeling of either the compound or the cancer marker (McConnell et al. Science 257:1906-1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and cancer markers.

In yet another embodiment, a cell-free assay is provided in which a cancer marker protein or biologically active portion thereof is contacted with a test compound and the ability of the test compound to bind to the stem cell cancer marker protein or biologically active portion thereof is evaluated. Biologically active portions of the cancer markers proteins to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in 15 the assay should be maximal. An FRET binding event can be conveniently measured through standard fluorometric detection means well known in the art (e.g., using a fluorimeter).

In another embodiment, determining the ability of the stem cell cancer markers protein to bind to a target molecule can be accomplished using real-time Biomolecular Interaction Analysis (BIA) (see, e.g., Sjolander and Urbaniczky, Anal. Chem. 63:2338-2345 [1991] and Szabo et al. Curr. Opin. Struct. Biol. 5:699-705 [1995]). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. The target gene product can be anchored onto a solid surface, and the test compound, (which is not anchored), can be labeled, either directly or indirectly, with detectable labels discussed herein.

It may be desirable to immobilize stem cell cancer markers, an anti-cancer marker antibody or its target molecule to facilitate separation of complexed from non-complexed forms of one or both of the proteins, as well as to accommodate automation of the assay. Binding of a test compound to a stem cell cancer marker protein, or interaction of a cancer marker protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-cancer marker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or cancer marker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of cancer markers binding or activity determined using standard techniques. Other techniques for immobilizing either cancer markers protein or a target molecule on matrices include using conjugation of biotin and streptavidin. Biotinylated cancer marker protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with stem cell cancer marker protein or target molecules but which do not interfere with binding of the stem cell cancer markers protein to its target molecule. Such antibodies can be derivatized to the wells of the plate, and unbound target or cancer markers protein trapped in the wells by antibody conjugation. Methods for detecting such complexes, in addition to those described above for the GST-immobilized complexes, include immunodetection of complexes using antibodies reactive with the cancer marker protein or target molecule, as well as enzyme-linked assays which rely on detecting an enzymatic activity associated with the cancer marker protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit. 11:141-8 [1998]; Hageand Tweed J. Chromatogr. Biomed. Sci. Appl 699:499-525 [1997]). Further, fluorescence energy transfer can also be conveniently utilized, as described herein, to detect binding without further purification of the complex from solution.

The assay can include contacting the stem cell cancer markers protein or biologically active portion thereof with a known compound that binds the cancer marker to form an assay mixture, contacting the assay mixture with a test compound, and determining the ability of the test compound to interact with a cancer marker protein, wherein determining the ability of the test compound to interact with a cancer marker protein includes determining the ability of the test compound to preferentially bind to cancer markers or biologically active portion thereof, or to modulate the activity of a target molecule, as compared to the known compound.

To the extent that stem cell cancer markers can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, cancer markers protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent WO 94/10300; each of which is herein incorporated by reference), to identify other proteins, that bind to or interact with cancer markers (“cancer marker-binding proteins” or “cancer marker-bp”) and are involved in cancer marker activity. Such cancer marker-bps can be activators or inhibitors of signals by the cancer marker proteins or targets as, for example, downstream elements of a cancer markers-mediated signaling pathway.

Modulators of cancer markers expression can also be identified. For example, a cell or cell free mixture is contacted with a candidate compound and the expression of cancer marker mRNA or protein evaluated relative to the level of expression of stem cell cancer marker mRNA or protein in the absence of the candidate compound. When expression of cancer marker mRNA or protein is greater in the presence of the candidate compound than in its absence, the candidate compound is identified as a stimulator of cancer marker mRNA or protein expression. Alternatively, when expression of cancer marker mRNA or protein is less (i.e., statistically significantly less) in the presence of the candidate compound than in its absence, the candidate compound is identified as an inhibitor of cancer marker mRNA or protein expression. The level of cancer markers mRNA or protein expression can be determined by methods described herein for detecting cancer markers mRNA or protein.

A modulating agent can be identified using a cell-based or a cell free assay, and the ability of the agent to modulate the activity of a cancer markers protein can be confirmed in vivo, e.g., in an animal such as an animal model for a disease or cells from a cancer resulting from metastasis of a pancreatic cancer (e.g., to a lymph node, bone, or liver), or cells from a pancreatic cancer cell line.

This invention further pertains to novel agents identified by the above-described screening assays (See e.g., below description of cancer therapies). Accordingly, it is within the scope of this invention to further use an agent identified as described herein (e.g., a cancer marker modulating agent, an antisense cancer marker nucleic acid molecule, a siRNA molecule, a cancer marker specific antibody, or a cancer marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein (e.g. to treat a human patient who has cancer).

In certain embodiments, the present invention employs non-adherent mammospheres for various screening procedures, including methods for screening c-Met and c-Met signaling pathway antagonists. Non-adherent mammospheres are an in vitro culture system that allows for the propagation of primary human mammary epithelial stem and progenitor cells in an undifferentiated state, based on their ability to proliferate in suspension as spherical structures. Non-adherent mammospheres have previously been described in Dontu et al Genes Dev. May 15, 2003; 17(10):1253-70, and Dontu et al., Breast Cancer Res. 2004; 6(6):R605-15, both of which are herein incorporated by reference. These references are incorporated by reference in their entireties and specifically for teaching the construction and use of non-adherent mammospheres. As described in Dontu et al., mammospheres have been characterized as being composed of stem and progenitor cells capable of self-renewal and multi-lineage differentiation. Dontu et al. also describes that mammospheres contain cells capable of clonally generating complex functional ductal-alveolar structures in reconstituted 3-D culture systems in Matrigel.

In certain embodiments, the following exemplary screening methods are employed. For in vitro studies, one could treat cells with either adenoviral constructs expressing control or c-Met candidate siRNA (m.o.i. 10 to 100) for 3 days or a small molecule candidate (e.g., PHA665752 derivative) (0.1-0.5 uM) for 3 days and compare the ability of c-Met+ cells to form tumor spheres compared in untreated vs. treated cells. For in vivo studies, one could infect human pancreatic cancer cells with a lentivirus expressing luciferase to monitor tumor growth. Luciferase-expressing cancer cells could be injected into the pancreas and tumors of approximately 0.5-0.7 cm in size could be established, with 5 animals per group. Animals with established tumors could then be treated with either a candidate c-Met inhibitor (daily i.v. 30 mg/kg/day for 7 days), or vehicle control. Parallel studies could be performed using infection with adenovirus expressing control or candidate c-Met siRNA (m.o.i. 100 or 500 for 7 days). Animals could be imaged at day 7, 14, 21, and 28 to assess tumor size and then be sacrificed. Tumor size could be further assessed at autopsy and a portion of the tumor stained to assess tumor histology. The remaining tumor could be harvested and sorted to assess the percentage of c-Met+ and c-Met negative cells. To verify that administration of candiate c-Met inhibitor and candidate c-Met siRNA adenovirus infection is inhibiting c-Met signaling function, phosphorylation of downstream mediators such as Gab-1 and ERK could be examined (see, Chistensen et al., Cancer Res., 2003; 63:7345-7355, herein incorporated by reference).

One could expect that HGF will enhance tumor sphere formation in c-Met positive pancreatic cancer stem cells and that silencing of c-Met using siRNA or blockade with a positive candidate c-Met inhibitor will inhibit the tumor sphere formation in vitro as well as tumor formation in vivo. One could also expect that the population of c-Met+ cells in the tumor will be significantly decreased following treatment. The doses of the small molecule candidate inhibitor and the adenoviral constructs one could use are similar to those shown to be effective in vitro and in vivo using other cancer cell systems (see, Hov et al., Clin. Cancer Res., Oct. 1, 2004:10(19):6686-94; Chistensen et al., and Shinomiya et al., Cancer Res. Nov. 1, 2004; 64(21):7962-70, all of which are herein incorporated by reference). In necessary, one could alter the dosing regimens outlined above to obtain blockade of c-Met in pancreatic cancer stem cells without undue toxicity.

### VI. Cancer Therapies

In some embodiments, the present invention provides therapies for cancer (e.g., pancreatic cancer). In some embodiments, therapies target cancer markers (e.g., including but not limited to, c-Met and proteins in the c-Met signaling pathway). In some embodiments, any known or later developed cancer stem cell therapy may be used. For example, cancer stem cell therapeutic agents are described in U.S. Pats. 6,984,522 and 7,115,360 and applications WO03/050502, WO05/074633, and WO05/005601, herein incorporated by reference in their entireties.

Antibody Therapy

In some embodiments, the present invention provides antibodies that target tumors that express a stem cell cancer marker of the present invention. Any suitable antibody (e.g., monoclonal, polyclonal, or synthetic) can be utilized in the therapeutic methods disclosed herein. In some embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against a stem cell cancer marker of the present invention, wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention can include a variety of cytotoxic moieties, including but not limited to, radioactive isotopes (e.g., iodine-131, iodine-123, technetium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments can include agents such as a coagulant, a cytokine, growth factor, bacterial endotoxin or the lipid A moiety of bacterial endotoxin. For example, in some embodiments, therapeutic agents will include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some embodiments, deglycosylated ricin A chain is utilized.

In any event, it is proposed that agents such as these can, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeted at stem cell cancer marker of the present invention. Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In some embodiments, antibody based therapeutics are formulated as pharmaceutical compositions as described below. In some embodiments, administration of an antibody composition of the present invention results in a measurable decrease in cancer (e.g., decrease or elimination of tumor).

### VII. Therapeutic Compositions and Administration

A pharmaceutical composition containing a regulator of tumorigenesis according the present invention can be administered by any effective method. For example, a c-Met or c-Met signaling pathway antagonist, or other therapeutic agent that acts as an antagonist of proteins in the c-Met signal transduction/response pathway can be administered by any effective method. In certain embodiments of the present invention, the Met targeting treatment is PHA-665752 [(3Z)-5-[(2,6-dichlorobenzyl)sulfonyl]-3-[(3,5-dimethyl-4-{[(2R)-2-(pyrro-lidin-1-ylmethyl)pyrrolidin-1-yl]carbonyl}-1H-pyrrol-2-yl)methylene]-1,3-dihydro-2H-indol-2-one (Pfizer, Inc., La Jolla, Calif.); Christensen et al., Cancer Research 63: 7345-7355, (2003)], SU11274 [Sattler et al., Cancer Research 63: 5462-5469 (2003)], or SU5416 [Fong et al., Cancer Research 59:99-106 (1999); Wang et al., J Hepatol 41: 267-273 (2004)]. In other embodiments, the Met targeting treatment is PF-02341066 (Pfizer, Inc.). In some embodiments, the c-Met inhibitor is as described in U.S. Patent Pub. 20050107391 or U.S. Pat. Pub. 20060246492, both of which are herein incorporated by reference in their entireties.

Other Met targeting treatments useful in the methods of the present invention include nucleic acid ligands to HGF and MET, such as those described in PCT International Publication No. WO 01/09159. Also encompassed in the present invention are MET antagonists described in U.S. Pat. Nos. 5,686,292 and 6,099,841, U.S. Pat. No. 6,174,889, PCT International Publication Nos. WO 00/43373, WO 98/07695 and WO 99/15550, all of which are herein incorporated by reference. Additional c-Met inhibitors include, but are not limited to, include, but are not limited to the following: indolinone hydrazides (WO05/005378), tetracyclic compounds (WO05/004808; U.S. published patent application No. 2005/0014755), arylmethyl triazolo and imidazopyrazines (WO05/004607), triazolotriazine compounds (WO05/010005), pyrrole compositions (WO05/016920), aminoheteroaryl compounds (WO04/076412; U.S. published patent application No. 2005/0009840), 4,6-diaminosubstituted-2-[oxy or aminoxy]-[1,3,5]triazines (WO04/031184), triarylimidazoles (U.S. published patent application No. 2005/0085473), 2-(2,6-dichlorophenyl)-diarylimidazoles (U.S. published patent application No. 2004/0214874; U.S. published patent application No. 2003/0199691), nucleic acids (U.S. published patent application No. 2003/0049644; U.S. Pat. No. 6,344,321; U.S. Pat. No. 5,646,036; WO01/09159), antibodies (U.S. Pat. No. 5,686,292; U.S. Pat. No. 5,646,036; U.S. published patent application No. 2004/0166544; U.S. published patent application No. 2005/0054019; WO05/016382; WO04/072117), peptide and polypeptides (U.S. Pat. No. 6,214,344; U.S. published patent application No. 2003/0118587; U.S. published patent application No. 2002/0136721 WO04/078778; WO05/030140) and others described in U.S. published patent application No. 2004/0210041; U.S. published patent application No. 2003/0118585; U.S. published patent application No. 2003/0045559; U.S. published patent application No. 2004/0185050.

Other MET inhibitors which can be used in the methods of the invention include but are not limited to the following: substituted 2,3-dihydro-1 h-isoindol-1-one derivatives (U.S. published patent application No. 2005/0054670); Geometrically restricted 3-cyclopentylidene-1,3-dihydroindol-2-ones (U.S. published patent application No. 2005/0038066), Substituted quinolinone derivatives (U.S. published patent application No. 2005/0049253), 5-sulfonamido-substituted indolinone compounds (U.S. published patent application No. 2004/0204407), Cyclic substituted fused pyrrolocarbazoles and isoindolones (U.S. published patent application No. 2004/0186157), Heterocyclic compounds (U.S. published patent application No. 2004/0209892), Indazolinone compositions (U.S. published patent application No. 2004/0167121), Heterocyclic substituted pyrazolones (U.S. published patent application No. 2003/0162775), Benzothiazole derivatives (U.S. published patent application No. 2003/0153568), Pyrazolopyrimidines (U.S. published patent application No. 2002/0156081) and others (U.S. published patent application No. 2004/0116388; U.S. published patent application No. 2004/0019067; U.S. published patent application No. 2003/0004174; U.S. published patent application No. 2003/0199534; U.S. published patent application No. 2002/0052386; U.S. published patent application No. 2003/0069430; U.S. published patent application No. 2004/0198750; U.S. published patent application No. 2004/0127453).

In certain embodiments, the c-Met inhibitor is an siRNA sequence. Exemplary siRNA sequence include the following:

| | |
|---|---|
| GACCTTCAGAAGGTTGCTG | (SEQ ID NO: 1) |
| GCCAGATTCTGCCGAACCA | (SEQ ID NO: 2) |
| GTGCAGTATCCTCTGACAG | (SEQ ID NO: 3) |

In certain embodiments, a physiologically appropriate solution containing an effective concentration of a c-Met signaling pathway antagonist can be administered topically, intraocularly, parenterally, orally, intranasally, intravenously, intramuscularly, subcutaneously or by any other effective means. In particular, the c-Met signaling pathway antagonist agent may be directly injected into a target cancer or tumor (e.g., into the pancreas) tissue by a needle in amounts effective to treat the tumor cells of the target tissue. Alternatively, a cancer or tumor present in a body cavity such as in the eye, gastrointestinal tract, genitourinary tract (e.g., the urinary bladder), pulmonary and bronchial system and the like can receive a physiologically appropriate composition (e.g., a solution such as a saline or phosphate buffer, a suspension, or an emulsion, which is sterile) containing an effective concentration of a c-Met signaling pathway antagonist via direct injection with a needle or via a catheter or other delivery tube placed into the cancer or tumor afflicted hollow organ. Any effective imaging device such as X-ray, sonogram, or fiber-optic visualization system may be used to locate the target tissue and guide the needle or catheter tube. In another alternative, a physiologically appropriate solution containing an effective concentration of a c-Met signaling pathway antagonist can be administered systemically into the blood circulation to treat a cancer or tumor that cannot be directly reached or anatomically isolated.

Such manipulations have in common the goal of placing the c-Met signaling pathway antagonist in sufficient contact with the target tumor to permit the c-Met signaling pathway antagonist to contact, transduce or transfect the tumor cells (depending on the nature of the agent). In one embodiment, solid tumors present in the epithelial linings of hollow organs may be treated by infusing the suspension into a hollow fluid filled organ, or by spraying or misting into a hollow air filled organ. Thus, the tumor cells (such as a solid tumor stem cells) may be present in or among the epithelial tissue in the lining of pulmonary bronchial tree, the lining of the gastrointestinal tract, the lining of the female reproductive tract, genitourinary tract, bladder, the gall bladder and any other organ tissue accessible to contact with the c-Met signaling pathway antagonist. In another embodiment, the solid tumor may be located in or on the lining of the central nervous system, such as, for example, the spinal cord, spinal roots or brain, so that the c-Met signaling pathway antagonist infused in the cerebrospinal fluid contacts and transduces the cells of the solid tumor in that space. One skilled in the art of oncology can appreciate that the c-Met signaling pathway antagonist can be administered to the solid tumor by direct injection into the tumor so that the c-Met signaling pathway antagonist contacts and affects the tumor cells inside the tumor.

The tumorigenic cells identified by the present invention can also be used to raise anti-cancer cell antibodies. In one embodiment, the method involves obtaining an enriched population of tumorigenic cells or isolated tumorigenic cells; treating the population to prevent cell replication (for example, by irradiation); and administering the treated cell to a human or animal subject in an amount effective for inducing an immune response to solid tumor stem cells. For guidance as to an effective dose of cells to be injected or orally administered; see, U.S. Pat. Nos. 6,218,166, 6,207,147, and 6,156,305, incorporated herein by reference. In another embodiment, the method involves obtaining an enriched population of solid tumor stem cells or isolated solid tumor stem cells; mixing the tumor stem cells in an in vitro culture with immune effector cells (according to immunological methods known in the art) from a human subject or host animal in which the antibody is to be raised; removing the immune effector cells from the culture; and transplanting the immune effector cells into a host animal in a dose that is effective to stimulate an immune response in the animal.

In some embodiments of the present invention, the anti-tumorigenic therapeutic agents (e.g. c-Met signaling pathway antagonists) of the present invention are co-administered with other anti-neoplastic therapies. A wide range of therapeutic agents find use with the present invention. Any therapeutic agent that can be co-administered with the agents of the present invention, or associated with the agents of the present invention is suitable for use in the methods of the present invention.

Various classes of antineoplastic (e.g., anticancer) agents are contemplated for use in certain embodiments of the present invention. Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

In some embodiments, exemplary anticancer agents suitable for use in compositions and methods of the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; and 23) radiation.

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and |
| (IgG1κ anti CD52 antibody) | | ILEX Partners, LP, |
| | | Cambridge, MA |
| Alitretinoin | Panretin | Ligand |
| (9-cis-retinoic acid) | | Pharmaceuticals, Inc., |
| | | San Diego CA |
| Allopurinol | Zyloprim | GlaxoSmithKline, |
| (1,5-dihydro-4 H-pyrazolo[3,4-d]pyrimidin-4-one | | Research Triangle |
| monosodium salt) | | Park, NC |
| Altretamine | Hexalen | US Bioscience, West |
| (N,N,N′,N′,N″,N″,-hexamethyl-1,3,5-triazine-2,4, | | Conshohocken, PA |
| 6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile,a,a,a′,a′-tetramethyl- | | Pharmaceuticals, LP, |
| 5-(1H-1,2,4-triazol-1-ylmethyl)) | | Wilmington, DE |
| Arsenic trioxide | Trisenox | Cell Therapeutic, |
| | | Inc., Seattle, WA |
| Asparaginase | Elspar | Merck & Co., Inc., |
| (L-asparagine amidohydrolase, type EC-2) | | Whitehouse Station, |
| | | NJ |
| BCG Live | TICE BCG | Organon Teknika, |
| (lyophilized preparation of an attenuated strain of | | Corp., Durham, NC |
| Mycobacterium bovis (Bacillus Calmette-Gukin | | |
| BCG], substrain Montreal) | | |
| bexarotene capsules | Targretin | Ligand |
| (4-[1-(5,6,7,8-tetrahydro-3,5,5,8,8-pentamethyl-2- | | Pharmaceuticals |
| napthalenyl) ethenyl] benzoic acid) | | |
| bexarotene gel | Targretin | Ligand |
| | | Pharmaceuticals |
| Bleomycin | Blenoxane | Bristol-Myers Squibb |
| (cytotoxic glycopeptide antibiotics produced by | | Co., NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0,0′]-,(SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford |
| | | Pharmaceuticals, Inc., |
| | | Baltimore, MD |
| Celecoxib | Celebrex | Searle |
| (as 4-[5-(4-methylphenyl)-3-(trifluoromethyl)- | | Pharmaceuticals, |
| 1H-pyrazol-1-yl] benzenesulfonamide) | | England |
| Chlorambucil | Leukeran | GlaxoSmithKline |
| (4-[bis(2chlorethyl)amino]benzenebutanoic acid) | | |
| Cisplatin | Platinol | Bristol-Myers Squibb |
| (PtCl₂H₆N₂) | | |
| Cladribine | Leustatin, 2-CdA | R. W. Johnson |
| (2-chloro-2′-deoxy-b-D-adenosine) | | Pharmaceutical |
| | | Research Institute, |
| | | Raritan, NJ |
| Cyclophosphamide | Cytoxan, Neosar | Bristol-Myers Squibb |
| (2-[bis(2-chloroethyl)amino] tetrahydro-2H-13,2- | | |
| oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye |
| | | Pharmaceuticals, Inc., |
| | | San Diego, CA |
| Dacarbazine | DTIC-Dome | Bayer AG, |
| (5-(3,3-dimethyl-1-triazeno)-imidazole-4- | | Leverkusen, |
| carboxamide (DTIC)) | | Germany |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., |
| (recombinant peptide) | | Thousand Oaks, CA |
| daunorubicin liposomal | DanuoXome | Nexstar |
| ((8S-cis)-8-acetyl-10-[(3-amino-2,3,6-trideoxy-á- | | Pharmaceuticals, Inc., |
| L-lyxo-hexopyranosyl)oxy]-7,8,9,10-tetrahydro- | | Boulder, CO |
| 6,8,11-trihydroxy-1-methoxy-5,12- | | |
| naphthacenedione hydrochloride) | | |
| Daunorubicin HCl, daunomycin | Cerubidine | Wyeth Ayerst, |
| ((1 S,3 S)-3-Acetyl-1,2,3,4,6,11-hexahydro- | | Madison, NJ |
| 3,5,12-trihydroxy-10-methoxy-6,11-dioxo-1- | | |
| naphthacenyl 3-amino-2,3,6-trideoxy-(alpha)-L- | | |
| lyxo-hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis |
| (2R,3S)-N-carboxy-3-phenylisoserine, N-tert- | | Pharmaceuticals, Inc., |
| butyl ester, 13-ester with 5b-20-epoxy- | | Bridgewater, NJ |
| 12a,4,7b,10b,13a-hexahydroxytax-11-en-9-one 4- | | |
| acetate 2-benzoate, trihydrate) | | |
| Doxorubicin HCl | Adriamycin, | Pharmacia & Upjohn |
| (8S,10S)-10-[(3-amino-2,3,6-trideoxy-a-L-lyxo- | Rubex | Company |
| hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin PFS | Pharmacia & Upjohn |
| | Intravenous | Company |
| | injection | |
| doxorubicin liposomal | Doxil | Sequus |
| | | Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo |
| | injection | Alto, CA |
| Elliott's B Solution | Elliott's B | Orphan Medical, Inc |
| | Solution | |
| Epirubicin | Ellence | Pharmacia & Upjohn |
| (8S-cis)-10-[(3-amino-2,3,6-trideoxy-a-L- | | Company |
| arabino-hexopyranosyl)oxy]-7,8,9,10-tetrahydro- | | |
| 6,8,11-trihydroxy-8-(hydroxyacetyl)-1-methoxy- | | |
| 5,12-naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3- | | Company |
| bis(2-chloroethyl)carbamate] 17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral | | Inc., Cedar Knolls, |
| agent vidarabine, 9-b-D-arabinofuranosyladenine | | NJ |
| (ara-A)) | | |
| Fluorouracil, 5-FU | Adrucil | ICN Pharmaceuticals, |
| (5-fluoro-2,4(1H,3H)-pyrimidinedione) | | Inc., Humacao, |
| | | Puerto Rico |
| Fulvestrant | Faslodex | IPR Pharmaceuticals, |
| (7-alpha-[9-(4,4,5,5,5-penta fluoropentylsulphinyl) | | Guayama, Puerto |
| nonyl]estra-1,3,5-(10)-triene-3,17-beta-diol) | | Rico |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| (acetate salt of [D-Ser(But)⁶,Azgly¹⁰]LHRH; pyro- | | Pharmaceuticals |
| Glu-His-Trp-Ser-Tyr-D-Ser(But)-Leu-Arg-Pro- | | |
| Azgly-NH2 acetate [C₅₉H₈₄N₁₈O₁₄•(C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal antibody | | |
| Ibritumomab and the linker-chelator tiuxetan [N- | | |
| 2-bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5,12-Naphthacenedione, 9-acetyl-7-[(3-amino- | | Company |
| 2,3,6-trideoxy-(alpha)-L-lyxo- | | |
| hexopyranosyl)oxy]-7,8,9,10-tetrahydro-6,9,11- | | |
| trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4-methyl- | | Switzerland |
| 3-[[4-(3-pyridinyl)-2-pyrimidinyl]amino]- | | |
| phenyl]benzamide methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperi- | | Company |
| dinopiperidino)carbonyloxy]-1H-pyrano[3′,4′: | | |
| 6,7] indolizino[1,2-b] quinoline-3,14(4H,12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl- | Leucovorin | Seattle, WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((−)-(S)-2,3,5,6-tetrahydro-6-phenylimidazo [2,1- | | Foundation, |
| b] thiazole monohydrochloride C₁₁H₁₂N₂S•HCl) | | Titusville, NJ |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6 H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7-one) | | Exton, Pa |
| Mitomycin C | Mutamycin | Bristol-Myers Squibb |
| mitomycin C | Mitozytrex | SuperGen, Inc., |
| | | Dublin, CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex |
| (1,4-dihydroxy-5,8-bis[[2-[(2- | | Corporation |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, |
| | | Germany |
| Oprelvekin | Neumega | Genetics Institute, |
| (IL-11) | | Inc., Alexandria, VA |
| Oxaliplatin | Eloxatin | Sanofi Synthelabo, |
| (cis-[(1R,2R)-1,2-cyclohexanediamine-N,N′] | | Inc., NY, NY |
| [oxalato(2-)-O,O′] platinum) | | |
| Paclitaxel | TAXOL | Bristol-Myers Squibb |
| (5β,20-Epoxy-1,2a,4,7β,10β,13a- | | |
| hexahydroxytax-11-en-9-one 4,10-diacetate 2- | | |
| benzoate 13-ester with (2R,3 S)-N-benzoyl-3- | | |
| phenylisoserine) | | |
| Pamidronate | Aredia | Novartis |
| (phosphonic acid (3-amino-1-hydroxypropylidene) | | |
| bis-, disodium salt, pentahydrate, (APD)) | | |
| Pegademase | Adagen | Enzon |
| (monomethoxypolyethylene glycol succinimidyl) | (Pegademase | Pharmaceuticals, Inc., |
| 11-17-adenosine deaminase) | Bovine) | Bridgewater, NJ |
| Pegaspargase | Oncaspar | Enzon |
| (monomethoxypolyethylene glycol succinimidyl | | |
| L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT |
| | | Phototherapeutics, |
| | | Inc., Vancouver, |
| | | Canada |
| Procarbazine | Matulane | Sigma Tau |
| (N-isopropyl-μ-(2-methylhydrazino)-p-toluamide | | Pharmaceuticals, Inc., |
| monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, |
| (recombinant peptide) | | Inc., |
| Rituximab | Rituxan | Genentech, Inc., |
| (recombinant anti-CD20 antibody) | | South San Francisco, |
| | | CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and b)- | | |
| D-glucopyranose and 220 mg citric acid | | |
| anhydrous) | | |
| Talc | Sclerosol | Bryan, Corp., |
| (Mg₃Si₄O₁₀(OH)₂) | | Woburn, MA |
| Tamoxifen | Nolvadex | AstraZeneca |
| ((Z)2-[4-(1,2-diphenyl-1-butenyl) phenoxy]-N,N- | | Pharmaceuticals |
| dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4-dien- | | |
| 17-oic acid [dgr]-lactone) | | |
| Thioguanine, 6-TG | Thioguanine | GlaxoSmithKline |
| (2-amino-1,7-dihydro-6 H-purine-6-thione) | | |
| Thiotepa | Thioplex | Immunex |
| (Aziridine, 1,1′,1″-phosphinothioylidynetris-, or | | Corporation |
| Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′,4′: 6,7] indolizino [1,2-b] | | |
| quinoline-3,14-(4H,12H)-dione | | |
| monohydrochloride) | | |
| Toremifene | Fareston | Roberts |
| (2-(p-[(Z)-4-chloro-1,2-diphenyl-1-butenyl]- | | Pharmaceutical |
| phenoxy)-N,N-dimethylethylamine citrate (1:1)) | | Corp., Eatontown, NJ |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody (I | | |
| 131 is a radioimmunotherapeutic antibody)) | | |
| Trastuzumab | Herceptin | Genentech, Inc |
| (recombinant monoclonal IgG₁ kappa anti-HER2 | | |
| antibody) | | |
| Tretinoin, ATRA | Vesanoid | Roche |
| (all-trans retinoic acid) | | |
| Uracil Mustard | Uracil Mustard | Roberts Labs |
| | Capsules | |
| Valrubicin, N-trifluoroacetyladriamycin-14- | Valstar | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[trifluoroacetyl)-amino-α-L-lyxo- | | |
| hexopyranosyl]oxyl]-2-naphthacenyl]-2-oxoethyl | | |
| pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In still further embodiments, the present invention provides compounds of the present invention (and any other chemotherapeutic agents) associated with targeting agents that are able to specifically target particular cell types (e.g., tumor cells). Generally, the therapeutic compound that is associated with a targeting agent, targets neoplastic cells through interaction of the targeting agent with a cell surface moiety that is taken into the cell through receptor mediated endocytosis.

Any moiety known to be located on the surface of target cells (e.g., tumor cells) finds use with the present invention. For example, an antibody directed against such a moiety targets the compositions of the present invention to cell surfaces containing the moiety. Alternatively, the targeting moiety may be a ligand directed to a receptor present on the cell surface or vice versa. Similarly, vitamins also may be used to target the therapeutics of the present invention to a particular cell.

As used herein, the term “targeting molecules” refers to chemical moieties, and portions thereof useful for targeting therapeutic compounds to cells, tissues, and organs of interest. Various types of targeting molecules are contemplated for use with the present invention including, but not limited to, signal peptides, antibodies, nucleic acids, toxins and the like. Targeting moieties may additionally promote the binding of the associated chemical compounds (e.g., small molecules) or the entry of the compounds into the targeted cells, tissues, and organs. Preferably, targeting moieties are selected according to their specificity, affinity, and efficacy in selectively delivering attached compounds to targeted sites within a subject, tissue, or a cell, including specific subcellular locations and organelles.

Various efficiency issues affect the administration of all drugs—and of highly cytotoxic drugs (e.g., anticancer drugs) in particular. One issue of particular importance is ensuring that the administered agents affect only targeted cells (e.g., cancer cells), tissues, or organs. The nonspecific or unintended delivery of highly cytotoxic agents to nontargeted cells can cause serious toxicity issues.

Numerous attempts have been made to devise drug-targeting schemes to address the problems associated with nonspecific drug delivery. (See e.g., K. N. Syrigos and A. A. Epenetos Anticancer Res., 19:606-614 (1999); Y. J. Park et al., J. Controlled Release, 78:67-79 (2002); R. V. J. Chari, Adv. Drug Deliv. Rev., 31:89-104 (1998); and D. Putnam and J. Kopecek, Adv. Polymer Sci., 122:55-123 (1995)). Conjugating targeting moieties such as antibodies and ligand peptides (e.g., RDG for endothelium cells) to drug molecules has been used to alleviate some collateral toxicity issues associated with particular drugs.

The compounds and anticancer agents may be administered in any sterile, biocompatible pharmaceutical carrier, including, but not limited to, saline, buffered saline, dextrose, and water. In some embodiments, the pharmaceutical compositions of the present invention may contain one agent (e.g., an antibody). In other embodiments, the pharmaceutical compositions contain a mixture of at least two agents (e.g., an antibody and one or more conventional anticancer agents). In still further embodiments, the pharmaceutical compositions of the present invention contain at least two agents that are administered to a patient under one or more of the following conditions: at different periodicities, at different durations, at different concentrations, by different administration routes, etc. In some embodiments, the c-Met signaling pathway antagonist is administered prior to the second anticancer agent, e.g., 0.5, 1, 2 3, 4, 5, 10, 12, or 18 hours, 1, 2, 3, 4, 5, or 6 days, 1, 2, 3, or 4 weeks prior to the administration of the anticancer agent. In some embodiments, the c-Mets signaling pathway antagonist is administered after the second anticancer agent, e.g., 0.5, 1, 2 3, 4, 5, 10, 12, or 18 hours, 1, 2, 3, 4, 5, or 6 days, 1, 2, 3, or 4 weeks after the administration of the anticancer agent. In some embodiments, the c-Met signaling pathway antagonist and the second anticancer agent are administered concurrently but on different schedules, e.g., the c-Met signaling pathway antagonist compound is administered daily while the second anticancer agent is administered once a week, once every two weeks, once every three weeks, or once every four weeks. In other embodiments, the c-Met signaling pathway antagonist is administered once a week while the second anticancer agent is administered daily, once a week, once every two weeks, once every three weeks, or once every four weeks.

Depending on the condition being treated, preferred embodiments of the present pharmaceutical compositions are formulated and administered systemically or locally. Techniques for formulation and administration can be found in the latest edition of “Remington's Pharmaceutical Sciences” (Mack Publishing Co, Easton Pa.). Suitable routes may, for example, include oral or transmucosal administration as well as parenteral delivery (e.g., intramuscular, subcutaneous, intramedullary, intrathecal, intraventricular, intravenous, intraperitoneal, or intranasal administration).

The present invention contemplates administering therapeutic compounds and, in some embodiments, one or more conventional anticancer agents, in accordance with acceptable pharmaceutical delivery methods and preparation techniques. For example, therapeutic compounds and suitable anticancer agents can be administered to a subject intravenously in a pharmaceutically acceptable carrier such as physiological saline. Standard methods for intracellular delivery of pharmaceutical agents are contemplated (e.g., delivery via liposome). Such methods are well known to those of ordinary skill in the art.

In some embodiments, the formulations of the present invention are useful for parenteral administration (e.g., intravenous, subcutaneous, intramuscular, intramedullary, and intraperitoneal). Therapeutic co-administration of some contemplated anticancer agents (e.g., therapeutic polypeptides) can also be accomplished using gene therapy reagents and techniques.

In some embodiments of the present invention, therapeutic compounds are administered to a subject alone, or in combination with one or more conventional anticancer agents (e.g., nucleotide sequences, drugs, hormones, etc.) or in pharmaceutical compositions where the components are optionally mixed with excipient(s) or other pharmaceutically acceptable carriers. In preferred embodiments of the present invention, pharmaceutically acceptable carriers are biologically inert. In preferred embodiments, the pharmaceutical compositions of the present invention are formulated using pharmaceutically acceptable carriers well known in the art in dosages suitable for oral administration. Such carriers enable the pharmaceutical compositions to be formulated as tablets, pills, capsules, dragees, liquids, gels, syrups, slurries, solutions, suspensions and the like, for respective oral or nasal ingestion by a subject.

Pharmaceutical preparations for oral use can be obtained by combining the active compounds with solid excipients, optionally grinding the resulting mixture, and processing the mixture into granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc.; cellulose such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

In preferred embodiments, dosing and administration regimes are tailored by the clinician, or others skilled in the pharmacological arts, based upon well known pharmacological and therapeutic considerations including, but not limited to, the desired level of therapeutic effect, and the practical level of therapeutic effect obtainable. Generally, it is advisable to follow well-known pharmacological principles for administrating chemotherapeutic agents (e.g., it is generally advisable to not change dosages by more than 50% at time and no more than every 3-4 agent half-lives). For compositions that have relatively little or no dose-related toxicity considerations, and where maximum efficacy (e.g., destruction of cancer cells) is desired, doses in excess of the average required dose are not uncommon. This approach to dosing is commonly referred to as the “maximal dose” strategy. In certain embodiments, the c-Met signaling pathway antagonist is administered to a subject at a dose of 1-40 mg per day (e.g. for 4-6 weeks). In certain embodiments, subject is administered a loading dose of between 15-70 mg of the c-Met signaling pathway antagonist. In certain embodiments, the subject is administered a loading dose of about 35-45 mg of the c-Met signaling pathway antagonist (e.g. subcutaneously), and then daily doses of about 10 mg (e.g. subcutaneously) for about 4-6 weeks.

Additional dosing considerations relate to calculating proper target levels for the agent being administered, the agent's accumulation and potential toxicity, stimulation of resistance, lack of efficacy, and describing the range of the agent's therapeutic index.

In certain embodiments, the present invention contemplates using routine methods of titrating the agent's administration. One common strategy for the administration is to set a reasonable target level for the agent in the subject. In some preferred embodiments, agent levels are measured in the subject's plasma. Proper dose levels and frequencies are then designed to achieve the desired steady-state target level for the agent. Actual, or average, levels of the agent in the subject are monitored (e.g., hourly, daily, weekly, etc.) such that the dosing levels or frequencies can be adjusted to maintain target levels. Of course, the pharmacokinetics and pharmacodynamics (e.g., bioavailability, clearance or bioaccumulation, biodistribution, drug interactions, etc.) of the particular agent or agents being administered can potentially impact what are considered reasonable target levels and thus impact dosing levels or frequencies.

Target-level dosing methods typically rely upon establishing a reasonable therapeutic objective defined in terms of a desirable range (or therapeutic range) for the agent in the subject. In general, the lower limit of the therapeutic range is roughly equal to the concentration of the agent that provides about 50% of the maximum possible therapeutic effect. The upper limit of the therapeutic range is usually established by the agent's toxicity and not by its efficacy. The present invention contemplates that the upper limit of the therapeutic range for a particular agent will be the concentration at which less than 5 or 10% of subjects exhibit toxic side effects. In some embodiments, the upper limit of the therapeutic range is about two times, or less, than the lower limit. Those skilled in the art will understand that these dosing consideration are highly variable and to some extent individualistic (e.g., based on genetic predispositions, immunological considerations, tolerances, resistances, and the like). Thus, in some embodiments, effective target dosing levels for an agent in a particular subject may be 1, . . . 5, . . . 10, . . . 15, . . . 20, . . . 50, . . . 75, . . . 100, . . . 200, . . . X %, greater than optimal in another subject. Conversely, some subjects may suffer significant side effects and toxicity related health issues at dosing levels or frequencies far less (1, . . . 5, . . . 10, . . . 15, . . . 20, . . . 50, . . . 75, . . . 100, . . . 200, . . . X %) than those typically producing optimal therapeutic levels in some or a majority of subjects. In the absence of more specific information, target administration levels are often set in the middle of the therapeutic range.

In preferred embodiments, the clinician rationally designs an individualized dosing regimen based on known pharmacological principles and equations. In general, the clinician designs an individualized dosing regimen based on knowledge of various pharmacological and pharmacokinetic properties of the agent, including, but not limited to, F (fractional bioavailability of the dose), Cp (concentration in the plasma), CL (clearance/clearance rate), Vss (volume of drug distribution at steady state) Css (concentration at steady state), and t½ (drug half-life), as well as information about the agent's rate of absorption and distribution. Those skilled in the art are referred to any number of well known pharmacological texts (e.g., Goodman and Gilman's, Pharmaceutical Basis of Therapeutics, 10th ed., Hardman et al., eds., 2001) for further explanation of these variables and for complete equations illustrating the calculation of individualized dosing regimes. Those skilled in the art also will be able to anticipate potential fluctuations in these variables in individual subjects. For example, the standard deviation in the values observed for F, CL, and Vss is typically about 20%, 50%, and 30%, respectively. The practical effect of potentially widely varying parameters in individual subjects is that 95% of the time the Css achieved in a subject is between 35 and 270% that of the target level. For drugs with low therapeutic indices, this is an undesirably wide range. Those skilled in the art will appreciate, however, that once the agent's Cp (concentration in the plasma) is measured, it is possible to estimate the values of F, CL, and Vss directly. This allows the clinician to effectively fine tune a particular subject's dosing regimen.

In still other embodiments, the present invention contemplates that continuing therapeutic drug monitoring techniques be used to further adjust an individual's dosing methods and regimens. For example, in one embodiment, Css data is used is to further refine the estimates of CL/F and to subsequently adjust the individual's maintenance dosing to achieve desired agent target levels using known pharmacological principles and equations. Therapeutic drug monitoring can be conducted at practically any time during the dosing schedule. In preferred embodiments, monitoring is carried out at multiple time points during dosing and especially when administering intermittent doses. For example, drug monitoring can be conducted concomitantly, within fractions of a second, seconds, minutes, hours, days, weeks, months, etc., of administration of the agent regardless of the dosing methodology employed (e.g., intermittent dosing, loading doses, maintenance dosing, random dosing, or any other dosing method). However, those skilled in the art will appreciate that when sampling rapidly follows agent administration the changes in agent effects and dynamics may not be readily observable because changes in plasma concentration of the agent may be delayed (e.g., due to a slow rate of distribution or other pharmacodynamic factors). Accordingly, subject samples obtained shortly after agent administration may have limited or decreased value.

The primary goal of collecting biological samples from the subject during the predicted steady-state target level of administration is to modify the individual's dosing regimen based upon subsequently calculating revised estimates of the agent's CL/F ratio. However, those skilled in the art will appreciate that early postabsorptive drug concentrations do not typically reflect agent clearance. Early postabsorptive drug concentrations are dictated principally by the agent's rate of absorption, the central, rather than the steady state, volume of agent distribution, and the rate of distribution. Each of these pharmacokinetic characteristics have limited value when calculating therapeutic long-term maintenance dosing regimens.

Accordingly, in some embodiments, when the objective is therapeutic long-term maintenance dosing, biological samples are obtained from the subject, cells, or tissues of interest well after the previous dose has been administered, and even more preferably shortly before the next planned dose is administered.

In still other embodiments, where the therapeutic agent is nearly completely cleared by the subject in the interval between doses, then the present invention contemplates collecting biological samples from the subject at various time points following the previous administration, and most preferably shortly after the dose was administered.

In certain embodiments, the ability of a c-Met inhibitor to effectively target solid tumor stem cells in combination with a standard chemotherapeutic may be assayed according to the exemplary protocol that follows. First, one could establish tumors from 5 different xenografts in the orthotopic tail location (10 animals per group), then treat with c-Met inhibitor PHA665752 (daily i.v. 30 mg/kg/day) in the absence of presence of the standard chemotherapeutic agent gemcitabine (100 mg/kg 2× per week). Treatment could be carried out for 4 weeks, and then half of each group could be sacrificed for evaluation. If combination is successful, it could be expected that the number/extent of metastases will be reduced. It is likely that the addition of gemcitabine will promote inhibition of tumor growth at the primary site when added to the c-Met inhibitor, but will have little/no effect on the formation of metastases. It is possible that the percentage of c-Met+ cells in the primary tumor may decrease but one may not see a change in primary tumor size. This may occur if blockade of c-Met affects only the cancer stem cell population, but has no immediate effect on the bulk of non-tumorigenic tumors at the primary site. One may instead find no significant change in primary tumor size but a decrease in the number of metastases.

### VIII. Transgenic Animals Expressing Cancer Marker Genes

The present invention contemplates the generation of transgenic animals comprising an exogenous cancer marker gene of the present invention (e.g., c-Met) or mutants and variants thereof (e.g., truncations or single nucleotide polymorphisms) or knock-outs thereof. In some embodiments, the transgenic animal displays an altered phenotype (e.g., increased or decreased presence of markers) as compared to wild-type animals. Methods for analyzing the presence or absence of such phenotypes include but are not limited to, those disclosed herein. In some embodiments, the transgenic animals further display an increased or decreased growth of tumors or evidence of cancer.

The transgenic animals of the present invention find use in drug (e.g., cancer therapy) screens. In some embodiments, test compounds (e.g., a c-Met inhibitor) and control compounds (e.g., a placebo) are administered to the transgenic animals and the control animals and the effects evaluated.

The transgenic animals can be generated by any suitable technique. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., 1985, PNAS 82:4438-4442). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, 1976, PNAS 73:1260). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., 1985, PNAS 82:6927). Transfection is easily and efficiently obtained by culturing the blastomeres on a monolayer of virus-producing cells (Stewart, et al., 1987, EMBO J., 6:383).

Alternatively, infection can be performed at a later stage. Virus or virus-producing cells can be injected into the blastocoele (Jahner et al., 1982, Nature 298:623). Most of the founders will be mosaic for the transgene since incorporation occurs only in a subset of cells that form the transgenic animal. Further, the founder can contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, 1995, Mol. Reprod. Dev., 40:386).

In other embodiments, the transgene is introduced into embryonic stem cells and the transfected stem cells are utilized to form an embryo. ES cells are obtained by culturing pre-implantation embryos in vitro under appropriate conditions (Evans et al., 1981, Nature 292:154; Bradley et al., 1984, Nature 309:255; Gossler et al., 1986, PNAS 83:9065; and Robertson et al., 1986, Nature 322:445). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes can also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science, 1988, 240:1468). Prior to the introduction of transfected ES cells into the blastocoel, the transfected ES cells can be subjected to various selection protocols to enrich for ES cells which have integrated the transgene assuming that the transgene provides a means for such selection. Alternatively, the polymerase chain reaction can be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### EXAMPLES

The following example is provided in order to demonstrate and further illustrate certain preferred embodiments and aspects of the present invention and is not to be construed as limiting the scope thereof.

### Example 1

### c-Met Identifies Pancreatic Cancer Stem Cells

This example describes the identification of c-Met as a pancreatic cancer stem cell marker.

### Materials and Methods

### Primary Tumor Specimen Implantation

Samples of human pancreatic adenocarcinomas were obtained within 30 minutes following surgical resection according to Institutional Review Board—approved guidelines. Tumors were suspended in sterile RPMI 1640 and mechanically dissociated using scissors and then minced with a sterile scalpel blade over ice to yield 2×2-mm pieces. The tumor pieces were washed with serum-free PBS before implantation. Eight-week-old male NOD/SCID mice were anesthetized using an i.p. injection of 100 mg/kg ketamine and 5 mg/kg xylazine. A 5-mm incision was then made in the skin overlying the midabdomen, and three pieces of tumor were implanted s.c. The skin incision was closed with absorbable suture. The mice were monitored weekly for tumor growth for 16 weeks.

### Preparation of Single Cell Suspensions of Tumor Cells

Before digestion with collagenase, xenograft tumors or primary human tumors were cut up into small pieces with scissors and then minced completely using sterile scalpel blades. To obtain single-cell suspensions, the resultant minced tumor pieces were mixed with ultrapure collagenase IV (Worthington Biochemicals, Freehold, N.J.) in medium 199 (200 units of collagenase per mL) and allowed to incubate at 37° C. for 2.5 to 3 h for enzymatic dissociation. The specimens were further mechanically dissociated every 15 to 20 min by pipetting with a 10-mL pipette. At the end of the incubation, cells were filtered through a 40-μm nylon mesh and washed with HBSS/20% fetal bovine serum (FBS) and then washed twice with HBSS.

### Flow Cytometry

Flow cytometry was performed on a FACSAria machine as described (Li et al., Cancer Res. Feb. 1, 2007; 67(3):1030-7, herein incorporated by reference for all purposes). Side scatter and forward scatter profiles were used to eliminate cell doublets. The antibodies used were anti-c-Met (FITC) (eBioscience) and anti-H2K (Southern Biotech) each at a dilution of 1:40. In all experiments using human xenograft tissue, infiltrating mouse cells were eliminated by discarding H2K (mouse histocompatibility class I) cells during flow cytometry. Dead cells were eliminated by using the viability dye DAPI. Implantation of sorted cells into either the subcutaneous or orthotopic location was as described (Li et al.).

### Tumorsphere Assays

Following FACS sorting, single cell suspensions were washed twice using HBSS. Cells were resuspended in culture media containing 1% N2 supplement (Gibco), 2% B27 supplement (Gibco), 1% Antibiotic-Antimycotic (Gibco), 20 ng/ml human FGF-2 (Sigma), and 20 ng/ml EGF (Gibco) and plated in 6-well, ultra-low attachment plates (Corning). Forty eight hours later, plates were analyzed for sphere forming colonies and were quantified using an inverted microscope (Leica). For subsequent passaging of spheres, cells were collected and dissociated with trypsin, sieved through 40 micron strainer, and cultured in ultra-low attachment 96-well plates. Visually identified, single sorted cells were placed in wells of 96 well plates and monitored for tumor sphere formation.

### Results:

Tumorigenic Potential of Sorted c-Met+ Pancreatic Cancer Cells

The identification of human pancreatic cancer stem cells based on the expression of the cell surface markers CD24, CD44, and ESA has recently been reported (Li et al.). Using a triple surface marker scheme to measure therapeutic efficacy of targeting pancreatic cancer stem cells, however, may be difficult to translate to the clinic. The present Example, however, has identified a single marker (c-met) with in vivo tumorigenicity results equal or superior to those seen with CD44+CD24+ESA+ cells (Table 2, n=3 different tumors, passage 1-2).

TABLE 2
Tumor formation ability of sorted pancreatic
cancer cells using cell surface markers

| Cell No. | 10,000 | 1,000 | 500 | 100 |
|---|---|---|---|---|
| c-Met+ | N/A | N/A | 8/12 | 7/12 |
| c-Met− | 1/12 | 0/12 | 0/12 | 0/12 |
| CD44+CD24+ESA+ | 10/12 | 10/12 | 7/12 | 6/12 |
| CD44−CD24−ESA− | 1/12 | 0/12 | 0/12 | 0/12 |

Cells were isolated by flow cytometry based on expression of the indicated makers and assayed for the ability to form tumors after injection into the subcutaneum of the flank of NOD/SCID mice at doses of 100, 500, 10³, and 10⁴ cells. The analysis was completed 16 weeks following injection. Data is expressed as number of tumors formed/number of injections.

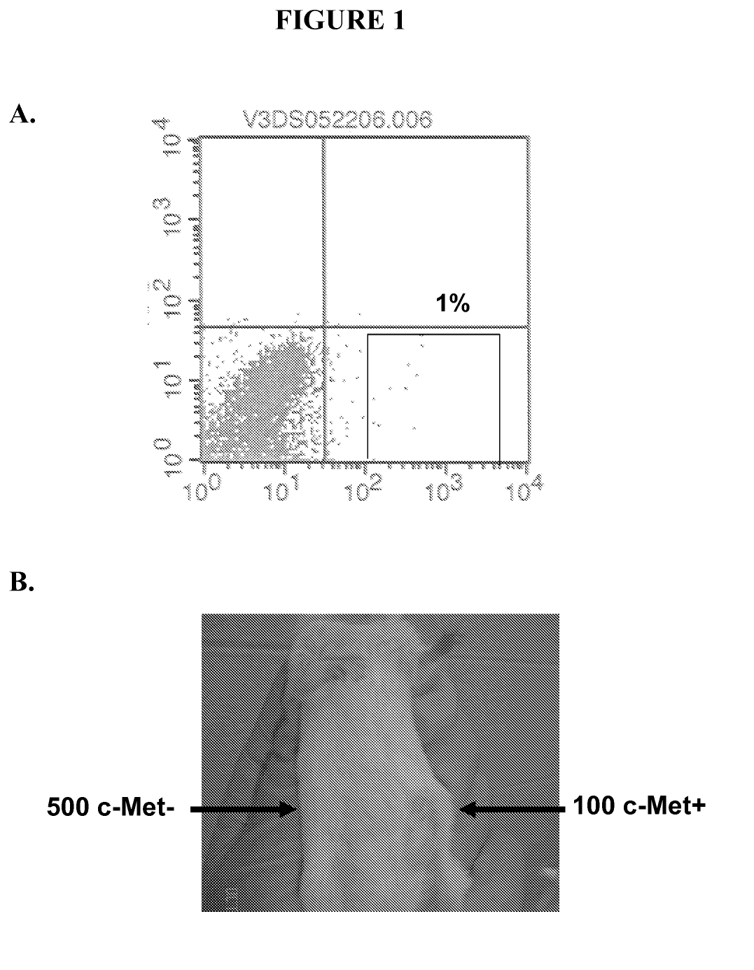

C-Met expressing cells represent 0.2-3% of all human pancreatic cancer cells and overlap 60% with CD44-expressing cells (data not shown). The percentage of cells expressing c-Met in primary tumors and xenografts derived from that tumor were similar. An example of c-Met sorted tumor cells from an individual patient is shown in FIG. 1A. Seven of twelve animals injected with as few as 100 c-Met+ cells formed tumors (Table 2, FIG. 1B), while cells negative for c-Met expression did not develop any tumors until 10⁴ cells were injected, and only 1/12 animals developed a tumor when injected with c-Met− cells (Table 2). These results show that the tumorigenic potential of c-Met+ population is slightly higher than the CD44+CD24+ESA+ population in the 3 tumors studied. The percentage of cancer cells expressing c-Met in individual tumors remained constant with passaging. The tumors derived from the c-Met+ pancreatic cancer cells appeared similar to histological sections of the primary human tumor (data not shown).

Development of Tumor Spheres from c-Met+ Cells

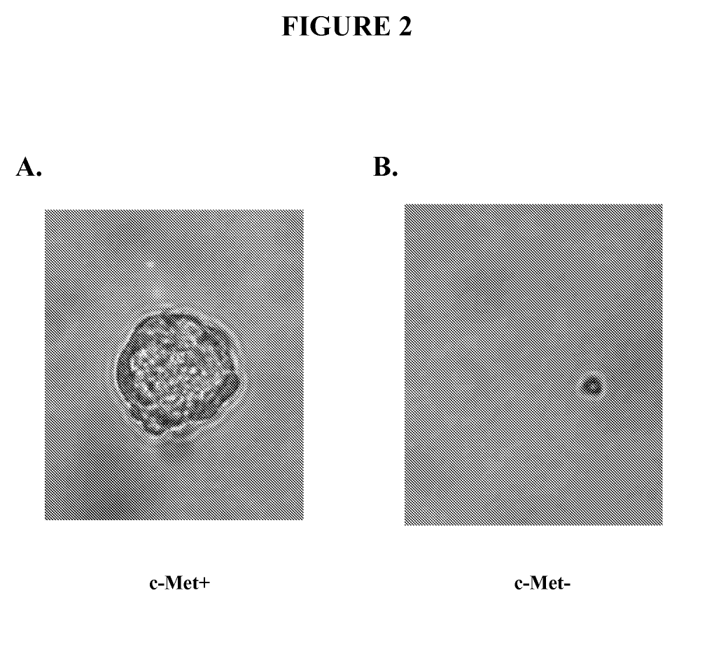

An in vitro approach to identify a cancer stem cell population is to culture single cells in a nonadhesive substratum. This assay measures self-renewal capacity and has been used successfully to identify cancer stem cells in brain, breast, and colon cancers. To test if c-Met+pancreatic cancer cells possess such stem-cell like features in vitro, single cells suspensions of c-Met+ and c-Met− cancer cells were generated and cultured in suspension. Tumor sphere formation from c-Met+ cells was routinely observed 5 days after plating, while no c-Met− cells formed tumor spheres (FIG. 2).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A method of detecting solid tumor stem cells comprising;
a) providing:
i) a tissue sample taken from a tumor of a subject, and
ii) an antibody, or antibody fragment, specific for the c-Met protein; and
b) contacting said tissue sample with said antibody, or antibody fragment, under conditions such that the presence or absence of c-Met+ solid tumor stem cells are detected.

2. The method of claim 1, wherein said antibody, or antibody fragment, is conjugated to a signal molecule.

3. The method of claim 2, wherein said signal molecule comprises a fluorescent molecule.

4. The method of claim 2, wherein said signal molecule comprises an enzyme that can catalyze a color producing reaction in the presence of a colorimetric substrate.

5. The method of claim 1, where said method further comprises contacting said sample with a secondary antibody, or secondary antibody fragment, specific for said antibody or antibody fragment.

6. The method of claim 5, wherein said secondary antibody, or secondary antibody fragment, comprises a signal molecule.

7. The method of claim 1, wherein no other proteins or nucleic acids are assayed in order to determine the presence or absence of said c-Met+ solid tumor stem cells.

8. The method of claim 1, wherein said tumor is a pancreatic tumor.

9-15. (canceled)

16. An isolated population of cancer stem cells that are:
a) tumorigenic; and
b) c-Met+.

17. The isolated population of claim 16, wherein said cancer stem cells are pancreatic cancer stem cells.

18. The isolated population of cancer stem cells of claim 16, wherein the population comprises at least 60% cancer stem cells and less than 40% non-tumorigenic tumor cells.

19. The isolated population of cancer stem cells of claim 16, wherein the cancer stem cells: are enriched at least two-fold compared to unfractionated non-tumorigenic tumor cells.

20-21. (canceled)

22. A method of treating a patient with pancreatic cancer comprising: administering a c-Met inhibitor to said patient such that the number of pancreatic stem cells expressing c-Met is reduced or eliminated in the subject.

23. The method of claim 22, wherein said method further comprises administering a chemotherapeutic selected from the group consisting of: 5-fluorouracil (5-FU); Gemcitabine (Gemzar); and Capecitabine

24. The method of claim 22, wherein said c-Met inhibitor is PHA-665752 or PF-02341066.

25. The method of claim 22, further comprising, prior to said administering, a step of contacting a sample from said patient comprising pancreatic tumor cells with a reagent that binds c-Met.

26-32. (canceled)
